(19) United States
(12) Patent Application Publication
(10) Pub. No.: US 2009/0093054 A1
(43) Pub. Date: Apr. 9, 2009

(54) CRYOPRESERVATION OF HUMAN BLASTOCYST-DERIVED STEM CELLS BY USE OF A CLOSED STRAW VITRIFICATION METHOD

(75) Inventors: Anita SJOGREN, Gothenburg (SE); Eva Karin Kilmare, Gothenburg (SE); Sven Enerback, Gothenburg (SE); Peter Eriksson, Gothenburg (SE)

Correspondence Address: BUCHANAN, INGERSOLL & ROONEY PC, POST OFFICE BOX 1404, ALEXANDRIA, VA, 22313-1404, US

(73) Assignee: Cellartis AB, Goteborg (SE)

(21) Appl. No.: 12/241,538

(22) Filed: Sep. 30, 2008

Related U.S. Application Data
(63) Continuation of application No. 10/555,411, filed on Feb. 9, 2006, now abandoned.
(60) Provisional application No. 60/469,320, filed on May 8, 2003.

(30) Foreign Application Priority Data
May 8, 2003 (DK) PA 2003 00700
Jun. 27, 2003 (DK) PA 2003 00983
Jun. 27, 2003 (DK) PA 2003 00984

(51) Int. Cl. C12N 5/08

(52) U.S. Cl. 435/366

(57) ABSTRACT

An improved method for vitrification of biological cells, especially blastocyst-derived stem cells (BS cells). The method is very mild for the cells that remain viable after they have been thawed. The method comprises, i) transfer of the cells to a first solution (solution A), ii) optionally incubation of the cells in the first solution, iii) transfer the cells obtained in step i) or ii) to a second solution (solution B), iv) optionally incubation of the cells in the second solution, v) transfer of the cells obtained from step iii) or iv) into one or more closed straws with dimensions that allow a volume of at least 20 μl to be contained in them vi) sealing the one or more closed straws, and vii) vitrification of the one or more closed straws. An important feature of the present invention is the use of closed straw and that relatively large volumes can be efficiently vitrified and subsequently thawed.

This application is a continuation application of application Ser. No. 10/555,441, filed Feb. 9, 2006, which is a national stage application, filed under 35 U.S.C. § 371, of PCT/EP2004/005031, filed on May 10, 2004, which claims priority to Danish Patent Application No. PA 2003 00700, filed May 8, 2003, Danish Patent Application No. PA 2003 00983, filed Jun. 27, 2003, and Danish Patent Application No. PA 2003 00984, filed Jun. 27, 2003, and U.S. Provisional Application No. 60/469,320, filed May 8, 2003, each of which is incorporated herein by reference in its entirety for all purposes.

### FIELD OF THE INVENTION

The present invention relates to an improved method for vitrification of biological cells, especially blastocyst-derived stem cells (BS cells). The method is very mild for the cells that remain viable after they have been thawed.

### BACKGROUND OF THE INVENTION

A stem cell is a cell type that has a unique capacity to renew itself and to give rise to specialized or differentiated cells. Although most cells of the body, such as heart cells or skin cells, are committed to conduct a specific function, a stem cell is uncommitted, until it receives a signal to develop into a specialized cell type. What makes the stem cells unique is their proliferative capacity, combined with their ability to become specialized. For years, researchers have focused on finding ways to use stem cells to replace cells and tissues that are damaged or diseased. So far, most research has focused on two types of stem cells, embryonic and somatic stem cells.

Embryonic stem cells are derived from the preimplanted fertilized oocyte, i.e. blastocyst, whereas the somatic stem cells are present in the adult organism, e.g. within the bone marrow, epidermis and intestine. According to many national laws in Europe and other countries, a fertilized oocyte is not regarded as an embryo before implantation in the uterus i.e. 10-14 days after fertilization, and such cells are therefore referred to as blastocyst-derived stem cells or hBS cells herein when employed according to the invention. Pluripotency tests have shown that whereas the embryonic or blastocyst-derived stem cells can give rise to all cells in the organism, including the germ cells, somatic stem cells have a more limited repertoire in descendent cell types.

In 1998, investigators were for the first time able to isolate embryonic stem cells from human fertilized oocytes and to grow them in culture see e.g. U.S. Pat. No. 5,843,780 and in U.S. Pat. No. 6,200,806.

The increasing research and development within the stem cell technology requires that suitable methods for preservation of the cells and cell lines are available. Cells may be stored either vitrified or frozen. Cryopreservation using conventional approaches is very difficult to apply to complex and sensitive biological material since the extracellular ice formation has destructive effects. By a vitrification process a sample containing the cells is rapidly cooled down to very low temperature and then the water content forms a glass-like state without crystallizing. Thus, vitrification is rapid cooling of a liquid medium in the absence of ice crystal formation. An amorphous glass forms during rapid cooling by direct submission into liquid nitrogen of e.g. a straw containing the cells. The glass retains the normal distribution of the liquid but remains in a supercooled form. The glass is devoid of ice crystals, and the cells are not subjected to cellular damage, which may be associated with ice crystal formation. Accordingly, vitrification is defined as solidification in an amorphous glassy state that obviates ice nucleation and growth.

Cryopreservation of human embryonic stem cells have been investigated and Reubinoff et al. (Human Reproduction, 2001,16,2187-2194) who described a method for cryopreservation of these cells by use of an open pulled straw vitrification method. The drawbacks of the method are that it involves contact of the open end of the straw with liquid nitrogen, which might be a source for contamination of the biological material to be vitrified. Furthermore, due to the dimensions of a pulled straw, the volumes vitrified by Reubinoff et al. were approximately 1 μl.

Methods for vitrification that avoid the direct contact with nitrogen have been described for rabbit embryos by Lopéz-Bejar et al. (Theriogenology, 2002, 58: 1541-52) and for mouse oocytes by Chen et al. (Human Reproduction, 2001, 16(11): 2350-56). Both of these methods use closed straws that have been pulled in the same way as the known open pulled straw and therefore possess the same dimensions as the straws used by Reubinoff et al. Thus, in both these methods a volume of about 1-2 μl is vitrified in each straw.

Slow-rate freezing and rapid thawing methods have been used for cryopreservation of cell lines. Although these methods are suitable for use for the cryopreservation of e.g. mouse embryonic stem cells, it seems that the survival of undifferentiated human embryonic stem cells is very poor, and most of the cell differentiate or die. Normally, larger volumes of cells have been vitrified with such slow-rate freezing methods resulting in low recovery (Reubinoff et al.).

Thus, there is still a need for developing effective vitrification methods that are easy to handle and that involves as few steps as possible, while at the same time avoid or at least reduce the risk of unwanted contamination of the cells during the procedures. In particular, there is a need for developing effective methods for the vitrification of larger volumes of cells or cell lines, such as hBS cells or hBS cell lines.

### DESCRIPTION OF THE INVENTION

As mentioned above, efficient cryopreservation methods are necessary for the development and widespread use of blastocyst-derived stem cell lines, hereunder the establishment of human blastocyst stem cell banks. Effective freezing and thawing techniques enable efficient preservation of cells and cell lines. For some purposes, it would be desirable to vitrify large volumes in each straw. This is the case e.g. when many cells are needed in a given procedure (or application) or when cells are to be dispatched by post in their vitrified state.

The present invention relates to a method for vitrification of cells, comprising

i) transfer of the cells to a first solution (solution A),
ii) optionally incubation of the cells in the first solution,
iii) transfer the cells obtained in step i) or ii) to a second solution (solution B),
iv) optionally incubation of the cells in the second solution,
v) transfer of the cells obtained from step iii) or iv) into one or more closed straws with dimensions that allow a volume of at least 20 μl to be contained in them
vi) sealing the one or more closed straws, and
vii) vitrification of the one or more closed straws.

A very important feature of the above-mentioned method is the large volume that can be vitrified in each straw. The present invention relates to a method for vitrification of cells in closed straws with dimensions that allow a volume from about 20 μl to about 250 μl, such as, e.g., from about 20 μl to about 225 μl, from about 25 μl to about to about 200 μl, from about 25 μl to about 175 μl, from about 25 μl to about 150 μl, from about 30 μl to about 125 μl, from about 30 μl to about 100 μl, from about 35 μl to about 75 μl, from about 40 to about 50 μl to be contained in them. The straws used in the provided examples of the present invention are approximately 13 cm long, a diameter of about 2 mm and a very thin plastic wall of about 0.1 mm (closed straws, French mini-straws, 250 μl, L'Aigle, IMV ZA 475°, 133 mm, Svensk Mjölk). However, it can be envisaged that even greater volumes can be successfully vitrified using longer straws as long as the diameter and the thickness of the straw is approximately the same as the straws used in herein, provided that the dimensions of the container with liquid nitrogen allows the entire length of the straw to be covered by liquid nitrogen.

Another very important step in the above-mentioned method is the use of so-called closed straw. In the present context, the term “closed straw” is used to denote straws that in the filling position have an open end to enable filling with the biological material (e.g. the cells or cell lines) e.g. in a suitable medium, but this end is immediately after filling tightly closed to avoid unwanted contamination of the cells from the surroundings and also to avoid the risk of unwanted contamination of the surroundings from the cells. Airtight seals on both ends of the straw are important to prevent contamination of both the samples and the environment. A suitable system is a Manual Sealing Unit called CBS SYMS from Cryo Bio System.

It is important that the straws are open from one side and have a stopper in the other side. This stopper allows air to be sucked with a syringe in order to fill the straw with liquids, but polymerizes once it gets in direct contact with a liquid, sealing the capillary at this end. Other suitable ways of sealing this end may also be applied. The other end will then be closed using a sealing (weld, bond, or the like). Important is that the wall is thin and the diameter is small which allows for rapid cooling of the content in the straw. The length is not so critical but for practical reasons it is good that is of standard length so it fits in standard holders in a liquid nitrogen tank. The straw is made of plastic but can be made of any suitable material including glass (although this might break easier). Important is that the material is safe and no substances can be absorbed or released, that it is non-porous, non-toxic, and biocompatible.

In the present application, the term “cryopreservation” denotes the preservation of biological material at an extremely low temperature.

The term “directly contacted” or “directly exposed” used in the present context mean that a biological material is “directly contacted” or “directly exposed” to e.g. a freezing material if a surface of the biological material or the medium, solution or material in which the material resides is allowed to come into contact with the freezing material.

The term “freezing material” as used in the present context, denotes any material that is capable of causing vitrification of a biological material. In theory any freezing media that is cold enough can be used since the samples are not in direct contact with it. Suitable materials include, but are not limited to, liquid gasses like liquid nitrogen, liquid propane, liquid helium, ethane or the like.

“Viable” used herein means that a biological material is able to live, develop and proliferate normally for a period of time.

According to the present invention, a volume of at least 20 μl biological material (e.g. hBS cells or hBS cell lines) is placed in closed straws. The closed straws are then exposed to a freezing material (suitably liquid nitrogen). Upon exposure to the freezing material, the cells undergo vitrification and can then be stored for a period of time and thawed at a later date. The thawed biological material remains viable. As it appears from the above, there is no direct contact or direct exposure of the cells and the freezing material. Thus, the risk for contamination of the cells (from external sources) as well as contamination of the environment with the cells is avoided.

The biological material of the present invention are living cells or cell lines especially BS cells, BS cells or cells derived from BS cells. The cells may be in any stage of development. Preferably, the cells are derived from an animal source including a mammalian source including, but not limited to humans, non-human primates such as monkeys, laboratory animal such as rats, mice and hamsters, agricultural livestock such as pigs, sheep, cows, goats and horses. In an especially interesting embodiment of the invention, the cells are human stem cells including human BS cells.

Suitable cells for use in a method according to the invention are BS cells or BS cell lines, especially hBS cells or hBS cell lines. The cells or cell lines may be obtained using the procedure described herein.

At least one of the vitrification solutions (the first and the second solution) may contain one or more cryoprotectants or mixtures of cryoprotectants. Non-toxic cryoprotectants are of course preferable. Cryoprotectants help minimizing shrinking by reducing the mole fraction of other solutes remaining in the non-frozen water. They inhibit the formation of crystalline ice, and thus depress the freezing point of the water. They may also prevent protein denaturation by hydrogen binding with bound water. As cells cool, solvent water converts to extracellular ice, and the increasing extracellular concentration of non-permeating electrolyte or non-electrolytes damages the cells. When treated with a cryoprotectant, cells do not reach the salt concentrations of non-treated cells until they reach much lower temperatures. Chemical reactions proceed very slowly at such low temperatures and consequently cellular damage is minimized. Usually it is better to use a combination of cryoprotectants since there might be differences between different types. The cryoprotectants may also function as osmotically active agents. Suitable cryoprotectants can be selected from the group consisting of ethylene glycol, propylene glycol, dimethylsulfoxide, glycerol, propane diol, sugars including sucrose, trehalose, maltose, lactose and methyl pentane diol. The concentration of the individual agents contained in the first and or the second solution is normally in a range of 5-50% v/v such as, e.g. from about 5% to about 40% v/v such as e.g. from about 5% to about 25% v/v (first solution) and from about 5% to about 30% v/v (second solution). Normally, the total concentration (i.e. calculated as v/v, w/v or M) of the cryoprotectant in the second solution is larger than that in the first solution. The first and the second solution may contain one or more cryoprotectants that are the same or different. The concentration of the one or more cryoprotectants in the first and the second solution can be the same or different, and normally the total concentration of the cryoprotectant in the second solution is larger than that in the first solution.

In a specific embodiment of the invention, the cryoprotectant is trehalose. The concentration of trehalose contained in the first and/or the second solution is normally within a range from about 0.02 M to about 1 M, such as, e.g., from about 0.05 M to about 0.9 M, from about 0.1 M to about 0.8 M, from about 0.2 M to about 0.7 M, from about 0.3 M to about 0.65 M, from about 0.4 M to about 0.6 M, from about 0.45 M to about 0.55 M. Usually, sucrose is used in similar applications. Trehalose is a unique, naturally occurring disaccharide and is found in hundreds of plants and animals. Trehalose is an important source of energy and has been shown to be a primary factor in stabilization of organisms during time of freezing. It has been shown that trehalose can depress the phase transition temperature of membranes so that they remain in the liquid-crystal state even when dry. Without being bound to any theory, it is hypothesized that this prevents membrane leakage during rehydration, thereby preserving cellular viability. With respect to proteins, trehalose has been shown to inhibit protein denaturation by exclusion of water from the protein surface when the cells are in the hydrated state.

In another embodiment of the invention, the cryoprotectant is sucrose. The concentration of sucrose contained in the first and/or the second solution is normally within a range from about 0.02 M to about 1 M, such as, e.g., from about 0.05 M to about 0.9 M, from about 0.1 M to about 0.8 M, from about 0.2 M to about 0.7 M, from about 0.3 M to about 0.65 M, from about 0.4 M to about 0.6 M, from about 0.45 M to about 0.55 M.

In yet another embodiment of the invention, at least one of the first and second solutions comprises a cryoprotectant.

At least one of the first and the second solution may comprise a viscosity-adjusting agent. Suitable viscosity-adjusting agent for use in the present context may be selected from the group consisting of Ficoll, Percoll, hyaluronic acid, albumin, polyvinyl pyrrolidone, alginic acid, gelatin and glycerol. The first and the second solution may contain one or more viscosity-adjusting agents that are the same or different. The concentration of the one or more viscosity-adjusting agents in the first and the second solution may be the same or different.

In a specific embodiment of the invention, the viscosity-adjusting agent is Ficoll. The concentration of Ficoll contained in the first and/or the second solution is at the most about 150 mg/ml, such as, e.g., at the most about 100 mg/ml, at the most about 50 mg/ml, at the most about 25 mg/ml, at the most about 15 mg/ml or at the most about 10 mg/ml.

In one embodiment of the invention, at least one of the first and second solutions is an aqueous solution.

In a specific embodiment of the invention, step ii) of the above-mentioned method is included.

A possible time span would be 10 sec-20 min since the point with this step is to promote equilibration with the solution and to ensure that the cryoprotectants sufficiently perfuses, but if DMSO is present the cells should not to be exposed to the somewhat toxic DMSO too long.

The incubation is normally performed at about 37° C. for a time period from between 5 sec to about 20 min such as, e.g., from about 10 sec to about 15 min, from about 15 sec to about 10 min, from about 20 sec to about 7.5 min, from about 30 sec to about 5 min, from about 40 sec to about 4 min, from about 50 sec to about 3 min, from about 30 sec to about 2 min, from about 45 sec to about 1.5 min or about 1 min.

In a further embodiment, step iv) is also included, and the incubation is normally performed at about 37° C. for a time period from between about 5 sec to about 10 min such as, e.g., from about 10 sec to about 7.5 min, from about 10 sec to about 5 min, from about 15 sec to about 4 min, from about 15 sec to about 3 min, from about 15 sec to about 2 min, from about 20 sec to about 1 min, from about 5 sec to about 1 min, from about 5 sec to about 30 sec or from about 10 sec to about 30 sec.

In a specific embodiment, step iv) is included and the incubation is performed at about 37° C. for about 30 sec or less.

The vitrification method is very efficient and mild to the cells. Normally, about 50% or more such as, e.g., about 55% or more, about 60% or more, about 65% or more, about 70% or more, about 75% or more, about 80% or more, about 85% or more, about 90% or more or about 95% or more of the cells are viable after being devitrified and cultures in a suitable medium. A suitable devitrification procedure is described herein.

In another aspect, the invention also relates to a cell, which has undergone vitrification by the method according to the invention.

In a further embodiment or in a separate aspect of the invention, it concerns a devitrification method.

The devitrification comprises

viii) subjecting one or more vitrified closed straw to an environment having a temperature of from about room temperature to about 40° C. for a time period of that allows the content of the closed straw to thaw,
ix) opening of the one or more closed straw,
x) subjecting the cells contained in the one or more opened closed straw to a washing procedure using a third solution (solution C),
xi) optionally transferring the washed cells obtained from step x) to a fourth solution (solution D), and
xii) optionally incubating the cells in the fourth solution,
xiii) optionally transferring the cells from xii) from the fourth solution and seeding the cells on feeder cells, and
xiv) optionally further cultivating the cells.

The cells obtained after step x) are ready to use for whatever purpose that is desired and the optional step may be applied in order to investigate the cells further e.g. for viability.

Step viii) concerns the thawing of the cells. The point here is just to thaw the content of the straw and this should be carried out during visual inspection of the straw, thus the timing is less important. The temperature should not be greater that 40° C. but can be between room temperature and 40° C. Higher temperatures could induce a heat shock when thawing the cells if they are not rapidly removed from the water bath after thawing.

Thus, the method may also—as mandatory steps—comprise steps xi), xiii) and xiv); and, furthermore, step xii).

The devitrification solutions may contain cryoprotectants e.g. cryoprotectants having osmotic activity such as osmotically active agents with low toxicity, generally avoiding e.g. DMSO, and preferentially using e.g. trehalose and sucrose, alone or in combination. The high percentage of the disaccharides in this solution prevents cellular disruption that otherwise would occur by the sudden contact with a solution without DMSO. The presence of the disaccharides outside the cells will prevent the natural osmotic force from acting and will allow enough time for the cells to discard the DMSO (or similar) present inside the cells and substitute it slowly by water.

Accordingly, the third and/or fourth (if relevant) solutions normally comprise one or more cryoprotectants.

In a specific embodiment, the one or more cryoprotectant is selected from the group consisting of glycerol, trehalose, sucrose, ethylene glycol, DMSO, propanediol, and or mixtures thereof, especially glycerol, trehalose, sucrose, or mixtures thereof is suitable for use.

The concentration of the cryoprotectant in the third and/or fourth solution is normally from about 0.02 M to about 1 M such as, e.g., from about 0.05 M to about 0.9 M, from about 0.1 M to about 0.8 M, from about 0.1 M to about 0.7 M, from about 0.1 M to about 0.6 M, from about 0.15 M to about 0.5 M, from about 0.2 M to about 0.4 M, and the concentration of the cryoprotectant in the third solution is larger than the concentration of the osmotically active agent in the fourth solution, if relevant. Normally, the concentration of the cryoprotectant in the third solution is larger than the concentration of the cryoprotectant in the fourth solution, if relevant.

In the following is given a general description of the method of the present invention.

Colonies of human blastocyst-derived stem (hBS) cells are cut into pieces (0.1-0.4 mm×0.1-0.4 mm). Up to 20 (preferably about 10) cell pieces in a volume of 40-50 μl can be frozen in a closed straw. A closed straw has a stopper in one end and is open in the other. After the cell pieces have been aspirated into the straw for freezing, the end with the plug (stopper) is sealed using cryo-PBS, while the open end is sealed using a bond (weld) and a Heatseal apparatus (Demtek, A/S). Before a larger amount of cells are frozen a test freezing and thawing round is performed. After thawing, the cell pieces are seeded onto a culture dish with mouse embryonic feeder cells (MEF). The human BS cells are cultured for one passage and are then evaluated.

All percentages mentioned are v/v.

The following description gives instructions on suitable procedures, solutions, time periods etc. However, based on the general description and guidance herein, a person skilled in the art may vary the different elements within the scope of the invention.

### Vitrification Procedure—General Description

### Preparations:

1. A stock solution consisting of 0.6M Trehalose in Cryo-PBS obtained from Vitrolife AB, Gothenburg, Sweden is prepared.
2.

| | |
|---|---|
| Solution A: | 10% Ethylene glycol is prepared in cryo-PBS and sterile |
| | filtered. |
| | Sterile DMSO is added to a final concentration of 10% |

3.

| | |
|---|---|
| Solution B: | A solution consisting of 0.3M Trehalose and 20% Ethylene |
| | glycol is prepared in cryo-PBS and sterile filtered. |
| | Sterile DMSO is added to a final concentration of 20% |

4. 1 ml of solution A and solution B respectively are placed in two separate wells in a 4-well plate (Nunclon, VWR International). The plate is place at 37° C.
5. Selected colonies of human blastocyst-derived stem cells, which display proper morphology, are cut in the same way as when the cells are cut for passage using an autoclaved drawn glass capillary (World Precision Instruments) (or a stem cell cutting tool from Swemed). The cutting tool from Swemed is a sterile sharpened glass capillary, with a 25 degree angle and a 200 or 300 micrometer lumen, designed for cutting, manipulation, and transfer of hBS colonies, or parts of hBS colonies. It is produced by Swemed Lab International AB, Billdal, Sweden.
6. The necessary number of straws (closed straws, French mini-straws, working volume of 250 μl, L'Aigle, IMV ZA 4750,133 mm, Svensk Mjölk) is labeled with the hBS-number and the visiotubes are labeled with the freezing code (i.e. cell line/date/signature).

### Freezing Procedure:

1. The cells that are to be frozen in a straw are transferred, using a glass capillary (World Precision Instruments) or a drawn glass pipette (Pasteur, VWR International), to Solution A.
2. The cells are incubated in Solution A for 1 min.
3. The cells are then transferred to one drop (25 μl) of Solution B, and within 25 s the cells are transferred to another fresh drop of solution B (25 μl).
4. The cells are incubated in Solution B for 25 s (maximum). The preferred time for point 3-4 should be as short as possible.
5. A 1-1.5 cm silicone tubing (autoclaved) is connected to a 1 ml syringe (tuberculin-syringe, single-use, Codan Triplus AB), which in turn is connected to the straw in the end with the cotton stopper (plugged end). The silicone tubing serves as a seal between the straw and the syringe. First, cryo-PBS is aspirated into the straw in an approximately 2-3 cm column. Approximately, 1-2 cm air is then aspirated (see FIG. 1).
6. The cells are then aspirated into the straw from solution B under a stereomicroscope in a 2 cm column.
7. Approximately 1-2 cm air is aspirated followed by 0.5-1 cm cryo-PBS (which serves as an extra stopper in that end).
8. The content of the straw is aspirated with the syringe so that the cryo-PBS comes in contact with the cotton stopper, which makes the stopper swell.
9. The straw is removed from the syringe using a pair of forceps and then sealed with a weld using a Heatseal apparatus.
10. The straw is placed in a visiotube, which in turn is placed in a tank containing liquid nitrogen for long term storage.

### Devitrification Procedure—General Procedure

### Preparations:

1. A stock solution consisting of 0.2M Trehalose in Cryo-PBS is prepared.
2. Solution C, 0.2M Trehalose in cryo-PBS is sterile filtered
3. Solution D: 0.1 M Trehalose in cryo-PBS is prepared and sterile filtered
4. 1 ml of solution C, solution D and hBS-medium (see below) respectively is placed in individual wells in a 4-well plate (Nunclon, VWR International) and the plate is incubated at 37° C. [The hBS medium contains KNOCKOUT® Dulbecco's Modified Eagle's Medium, supplemented with 20% KNOCKOUT® Serum replacement and the following constituents at their respective final concentrations: 100 units/ml penicillin, 0.1 mM non-essential amino acids, 2 mM L-glutamine, 100 μM β-mercaptoethanol, 4 ng/ml human recombinant bFGF (basic fibroblast growth factor).]

It is important to wash the thawed cells quickly from the DMSO, which was used in the vitrification solution. The presence of the less toxic trehalose contributes to a relatively slow step-wise change from vitrifying solution to the media used for seeding the cells. The concentrations can also be varied (5-50% v/v, w/w or w/v) with different efficiencies. It would also be possible to use other cryoprotectants with low toxicity.

### Thawing

1. Closed Straws containing vitrified human BS cells are collected from a storage tank containing liquid nitrogen and are placed in a vial containing liquid nitrogen.
2. The closed straw is held in the air at room temperature for 10 s and is then placed in a water bath at 40° C. for about 2 sec. The straw is dried using an autoclaved “all purpose rag” (allduk).
3. Using an autoclaved pair of scissors the plugged end of the straw is cut open just by the sealing. The straw is attached on a syringe using a piece of silicon tubing as sealing. The straw is then cut open at the bond (weld). The air in the syringe is used to push out the hBS cells into solution C. The amount of cells to be washed in the same well should not exceed the amount contained in one single straw.
4. The hBS cell colonies are incubated in solution C for 1 min. Normally from about 1 min to about 20 min.
5. Under a stereo-microscope, the hBS cell colonies are transferred to solution D using a glass capillary (World Precision Instruments) or a drawn glass pipette (Pasteur, VWR International).
6. The hBS cell colonies are incubated in solution D for 5 min. Normally from about 5 min to about 30 min.
7. Under a stereo-microscope, the hBS cell colonies are transferred to hBS-medium using a glass capillary (World Precision Instruments) or a drawn glass pipette (Pasteur, VWR International).
8. The colonies are within a few seconds (>5 s) transferred from the hBS-medium using a glass capillary (World Precision Instruments) or a drawn glass pipette (Pasteur, VWR International) and are seeded in a culture dish on top of mouse embryonic feeder cells (MEF).
9. The culture dish is placed in an incubator for further cultivation.

### FIGURES

FIG. 1: Thawing recovery after vitrification and devitrification, human BS cell line SA001.

FIG. 2: Thawing recovery after vitrification and devitrification, human BS cell line SA002.

FIG. 3: Thawing recovery after vitrification and devitrification, human BS cell line AS034.

FIG. 4 (A)-(C): Typical morphology of human BS cell line SA001 cultured on mouse embryonic feeder cells just prior to vitrification.

FIG. 5 (A)-(C): Typical morphology of human BS cell line SA001 cultured on mouse embryonic feeder cells at the first passage after devitrification.

FIG. 6: Typical morphology of human BS cell line SA001 after devitrification cultured on mouse embryonic feeder cells in passage 18 (A), passage 23 (B), passage 29 (C), and passage 35 (D).

FIG. 7: (A) human BS cell colony [p19], (B) SSEA-1 [p31], (C) SSEA-3 [p31], (D) SSEA-4 [p31], (E) TRA-1-60 [p31], (F) TRA-1-81 [p31], (G) Oct-4 [p31], (H) ALP [p31]

FIG. 8: Karyotype, cell line SA001 after vitrification

FIG. 9: In vitro differentiation of devitrified human BS cells (SA001), passage 29. (A) β-III-tubulin, (B) desmin, (C) α-fetoprotein, (D) HNF-3β

FIG. 10: In vivo differentiation of human BS cells (SA001), passage 19. (A) Endoderm (secretory epithelium), (B) Mesoderm (cartilage), (C) Ectoderm (neuroectoderm).

FIG. 11: Syringe with a closed straw prepared for freezing

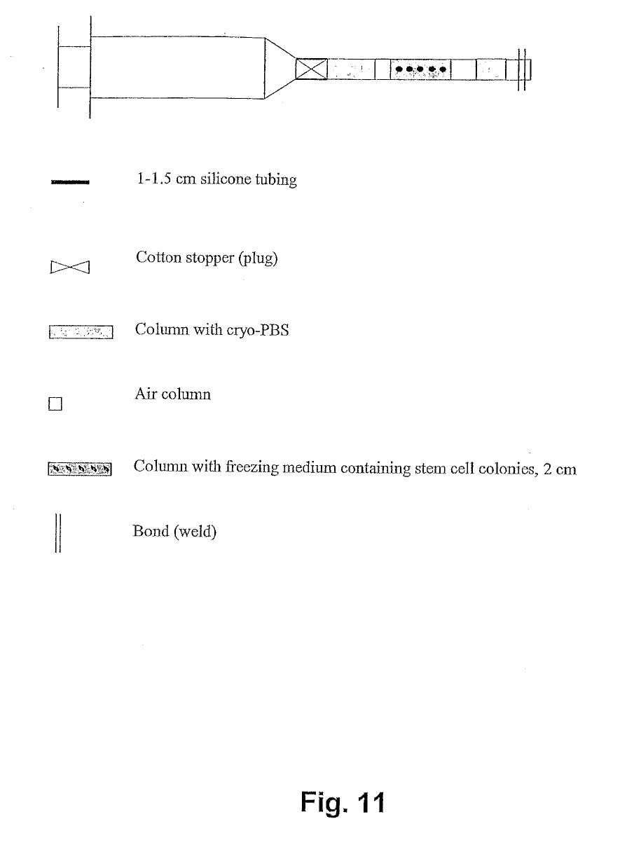

The invention is further illustrated in the following examples, which are not intended to limit the invention in any way.

### EXAMPLE 1

### Vitrification of hBS Cells

Two solutions A and B are prepared (Solution A: Sterile filtered 10% Ethylene glycol, 10% DMSO in Cryo-PBS; Solution B: Sterile filtered 0.3M Trehalose, 20% Ethylene glycol, 10% DMSO in Cryo-PBS) Selected colonies of human blastocyst-derived stem cells, which display proper morphology, are cut in the same way as when the cells are cut for regular passage using a stem cell cutting tool (Swemed Labs International, BilIdal, Sweden). The cell pieces should be about 0.1-0.4 mm×0.1-0.4 mm in size. The cell pieces are incubated first in 500 μl preheated (37° C.) Solution A for 1 min and then transferred to 25 μl Solution B and incubated for 30 s and then transferred again to a fresh drop of Solution B and incubated for 30 s. The volume is about 40-50 μl. About 10 cell pieces are aspirated into a straw prepared for vitrification and the straw is then closed with a bond. The straw is plunged into liquid nitrogen.

### EXAMPLE 2

### Thawing of Vitrified Human BS Cells

Two solutions C and D are prepared (Solution C: Sterile filtered 0.2M Trehalose in Cryo-PBS.; Solution D: Sterile filtered 0.1 M Trehalose in Cryo-PBS). Solutions C and D and hBS-medium are preheated at 37° C. A closed straw containing vitrified hBS cells (about 10 cell pieces) is removed from the liquid nitrogen tank. The straw is keep at room temperature for 10 s and then quickly thawed in a 40° C. water bath (within seconds). The straw is cut open in the plugged end using an autoclaved pair of scissors and the content pushed out from the straw into solution C using a syringe. The hBS cells are incubated for 1 min in 500 μl solution C and the transferred to 500 μl solution D and incubated for 5 min. Under a stero-microscope the hBS cell pieces are quickly rinsed in hBS medium and then seeded in a culture dish on top of mouse embryonic feeder cells in hBS medium. The cells are then cultured (incubated at 37° C.) and the number of established new colonies are counted and passaged in order to verify the viability of the hBS cells after vitrification. The recovery of viable cells following this vitrifying and thawing procedures is normally in the range of 70-100%.

### EXAMPLE 3

### Vitrification and Thawing of Human BS Cells using Closed Staws

Human BS cells (cell lines SA002, SA121, and SA181) were vitrified and thawed following the procedure described in Example 1 & 2. Forty-eight hours after seeding in culture dishes on top of mouse embryonic feeder cells the hBS cell colonies were evaluated and counted. The thawing recovery was calculated as the ratio between the number of viable thawed colonies (displaying appropriate hBS cell morphology) and the number of hBS cell pieces originally vitrified, since each of these cell pieces can give rise to one colony. Three straws were prepared and evaluated per cell line and the results are presented below for each individual straw, showing a recovery of between 40% and 100%.

Human BS cell line

| Closed straws | SA002 | SA181 |
|---|---|---|
| Thawing recovery | 3/7 | 9/9 |
| | 5/10 | 6/8 |
| | 10/10 | 2/5 |

### EXAMPLE 4

### Direct Comparison Between Closed Straws and Open Pulled Straws

Human BS cells (cell line AS034) were vitrified and thawed following the procedure described in Example 1 & 2 with the exception that open pulled straws were used in parallel to closed straws. Notably, only approximately 4 BS cell pieces can be vitrified in each open pulled straw. Forty-eight hours after seeding in culture dishes on top of mouse embryonic feeder cells the hBS cell colonies were evaluated and counted. The thawing recovery was calculated as the ratio between the number of viable thawed colonies (displaying appropriate hBS cell morphology) and the number of hBS cell pieces originally vitrified. Three straws were prepared and evaluated per cell line and the results are presented below for each individual straw and show the achievement of obtaining more viable cells from each of the closed straws (in absolute numbers) while maintaining an acceptable recovery.

Human BS cell line AS034

| | | Open pulled |
|---|---|---|
| | Closed straws | straws |
| Thawing recovery | 6/9 | 4/4 |
| | 6/9 | 2/4 |
| | 6/10 | 3/4 |

### EXAMPLE 5

### Comparison Between Using Trehalose and Sucrose in the Vitrification Medium

Human BS cells (cell line SA121) were vitrified and thawed either following the procedure described in Example 1 & 2 or following the procedure described in Example 1 and 2 with the exception that trehalose was used in the vitrification and devitrification medium was replaced by sucrose in the same molar concentration as used for trehalose. Forty-eight hours after seeding in culture dishes on top of mouse embryonic feeder cells the hBS cell colonies were evaluated and counted. The thawing recovery was calculated as the ratio between the number of viable thawed colonies (displaying appropriate hBS cell morphology) and the number of hBS cell pieces originally vitrified. Three straws were prepared and evaluated per cell line and the results are presented below for each individual straw and shows that in this case there seems to be no significant difference between using trehalose and sucrose.

Human BS cell line SA121

| | Trehalose | Sucrose |
|---|---|---|
| Thawing recovery | 5/9 | 8/10 |
| | 8/10 | 5/10 |
| | 6/10 | 9/10 |

### EXAMPLE 6

### Vitrification and Thawing of Human BS Cells using Ficoll in the Vitrification Medium

Human BS cells (cell lines SA121) were vitrified and thawed following the procedure described in Example 1 & 2 with the exception that Ficoll was used in the vitrification medium (0 mg/ml, 10 mg/ml, and 100 mg/ml). Forty-eight hours after seeding in culture dishes on top of mouse embryonic feeder cells the hBS cell colonies were evaluated and counted. The thawing recovery was calculated as the ratio between the number of viable thawed colonies (displaying appropriate hBS cell morphology) and the number of hBS cell pieces originally vitrified. Three straws were prepared and evaluated per cell line and the results are presented below for each individual straw.

| | Human BS cell line SA121 | | |
|---|---|---|---|
| | Ficoll concentration | | |
| Closed straws | 0 mg/ml | 10 mg/ml | 100 mg/ml |
| Thawing recovery | 5/5 | 7/10 | 6/10 |
| | 9/9 | 6/10 | 3/10 |
| | 6/10 | 3/10 | — |

### EXAMPLE 7

Comparison Between Using Different Concentrations of Trehalose in the Vitrification Medium

Human BS cells (cell line SA121) were vitrified and thawed following the procedure described in Example 1 & 2 with the exception that trehalose was used in two different concentrations (0.3M and 0.5M) in the second vitrification solution (solution B). When 0.3 M trehalose was used in solution B, solution C contained 0.2 M trehalose and solution D contained 0.1 M trehalose. When 0.5 M trehalose was used in solution B, solution C contained 0.4 M trehalose and solution D contained 0.2 M trehalose. Forty-eight hours after seeding in culture dishes on top of mouse embryonic feeder cells the hBS cell colonies were evaluated and counted. The thawing recovery was calculated as the ratio between the number of viable thawed colonies (displaying appropriate hBS cell morphology) and the number of hBS cell pieces originally vitrified. Two separate experiments using two different human BS cell lines were performed. Three straws were prepared and evaluated per cell line and the results are presented below for each individual straw. The results show that 0.5 M trehalose in solution B seems to work better than 0.3 M trehalose in solution B, although both of the investigated trehalose conditions work well.

Human BS cell line SA121 and SA002
Trehalose

| Closed straws | 0.3M | 0.5M |
|---|---|---|
| Thawing recovery | 6/10 | 8/10 |
| (SA121) | 5/10 | 9/10 |
| | 6/10 | 8/8 |
| Thawing recovery | 5/8 | 7/8 |
| (SA002) | 6/8 | 8/8 |
| | 7/7 | 5/7 |

### EXAMPLE 8

### Extensive Evaluation of Vitrification and Devitrification Procedure Using Closed Straws

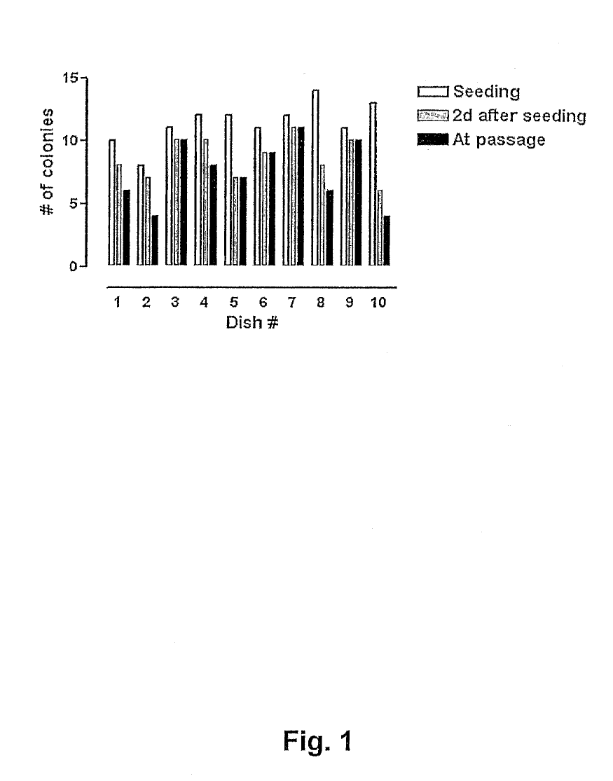

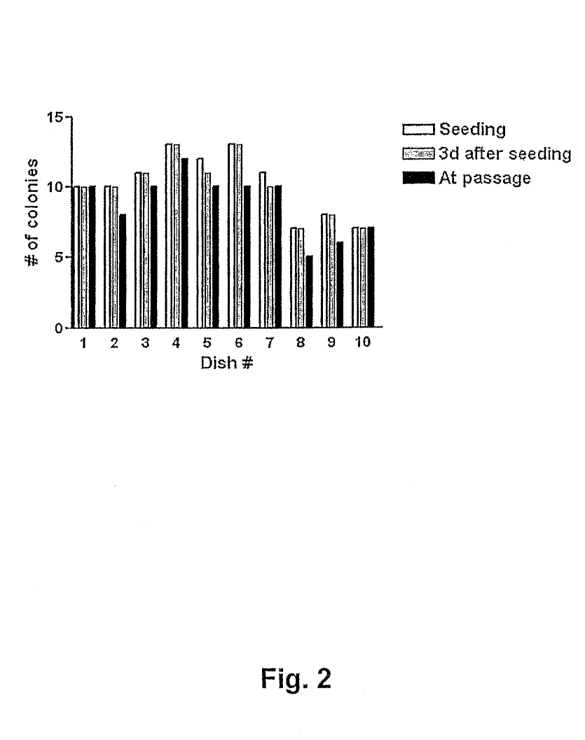

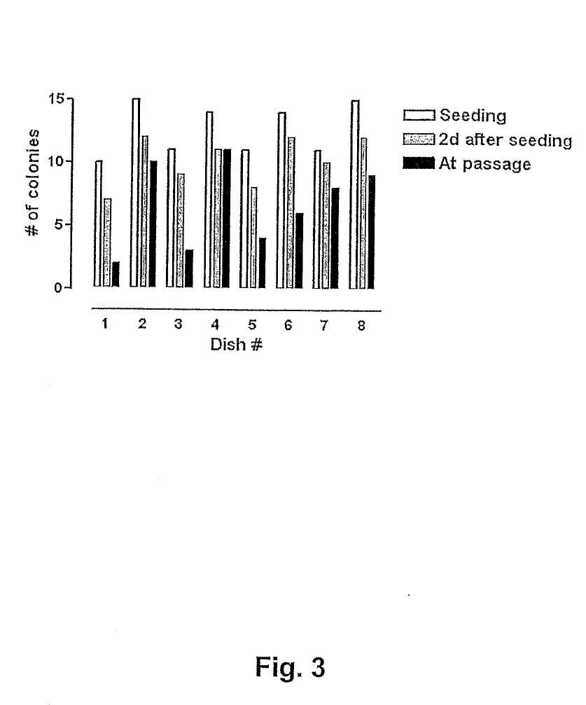

In order to evaluate the quality of the vitrification process large quantities of human BS cells were vitrified (as described in Example 1 above) at three different occasions using three different human BS cell lines (SA001, SA002, and AS034). At each occasion >100 straws were vitrified from each cell line. Eight to 10 straws each from of these large batches were randomly selected, devitrified (as described in Example 2 above) and seeded in separated dishes on top of mouse embryonic feeder cells. The number of hBS cell clumps that were seeded and that attached, proliferated, and displayed appropriate morphology was determined in each dish. The results are presented in FIGS. 1, 2 and 3 and show that every straw gave rise to viable hBS cell colonies that subsequently were passaged according to standard procedures and characterized.

### EXAMPLE 9

### Typical Morphology of Human BS Cell Before and after Vitrification and Thawing

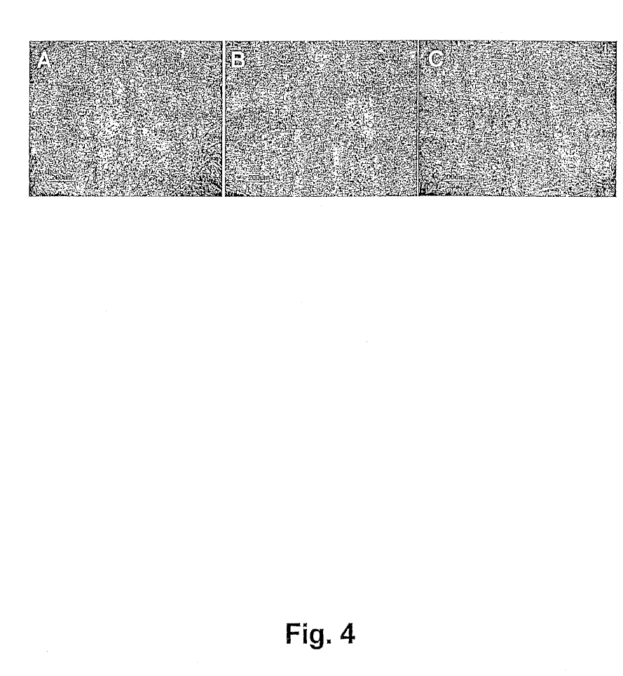

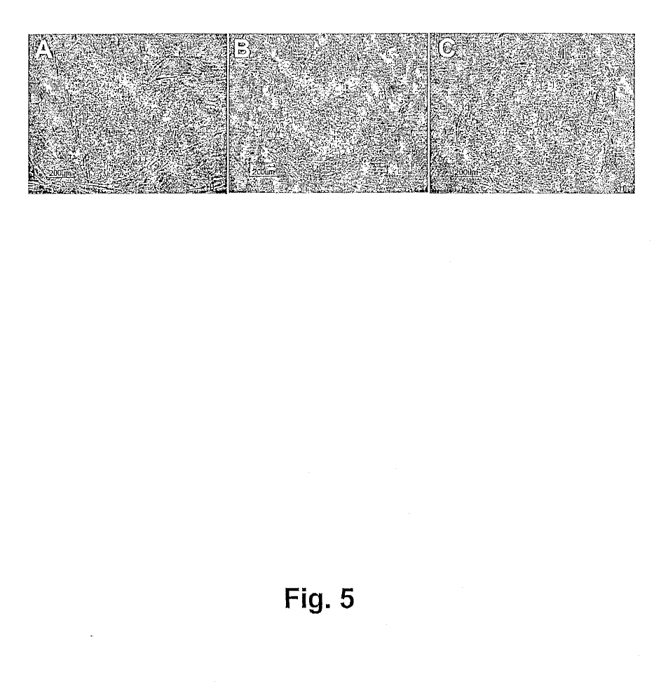

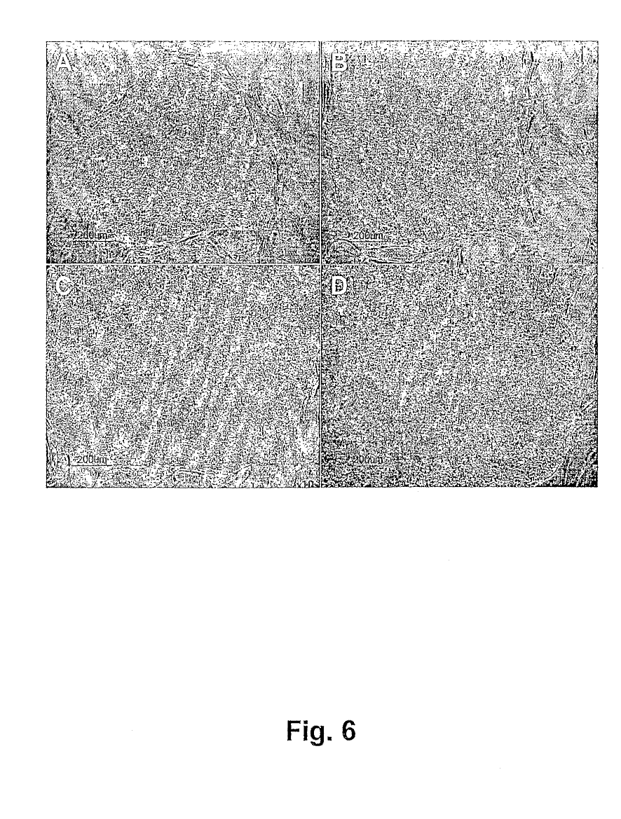

Typical morphology of the human BS colonies (cell line SA001) before vitrification is shown in FIG. 4. After devitrification and seeding, viable colonies proliferated and displayed morphology characteristic for undifferentiated human BS cells (FIG. 5). Subsequently, these cells were propagated and passaged according to standard procedures and representative illustrations of the human BS cell colonies are shown in FIG. 6. Similar results were obtained for human BS cell line SA002 and AS034 (data not shown).

### EXAMPLE 10

### Subsequent Characterization of Human BS Cells Subjected to Vitrification and Devitrification in Closed Straws

In order to verify that the human BS cells completely recover and display the proper characteristics after the vitrification and devitrification process, the hBS cells subjected to extensive characterization. This includes analysis of surface antigen expression, karyotyping, and pluripotency tests in vitro as well as in vivo. The results below were obtained using human BS cell line SA001, and similar results were also obtained using human BS cell lines SA002 and AS034 (data not shown).

### Immunohistochemical Staining of Undifferentiated hBS Cells

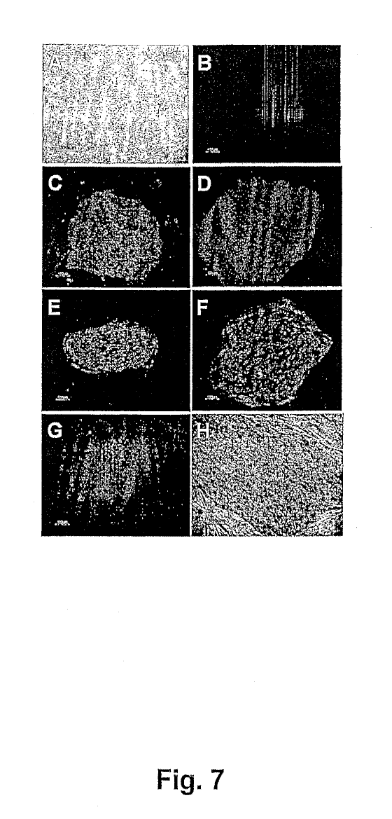

Devitrified human BS cells (cell line SA001) cultured on mouse embryonic feeder (MEF) cells were fixed in PFA and subsequently permeabilized using Triton X-100. After consecutive washing and blocking steps, the cells were incubated with the primary antibody (as indicated). Conjugated secondary antibodies were subsequently used for detection. The nuclei were visualized by DAPI staining. The activity of alkaline phosphatase (ALP) was determined using a commercial available kit following the instructions indicated by the manufacturer (Sigma Diagnostics, Stockholm, Sweden). The passage number at which each analysis was performed is indicated within brackets in the figure legend to FIG. 7. As illustrated in FIG. 7, the results show that the human BS cells displayed positive staining for SSEA-3, SSEA-4, TRA-1-60, TRA-1-81, Oct-4, and ALP and that they were negative for SSEA-1 as expected for undifferentiated human BS cells.

### Karyotyping

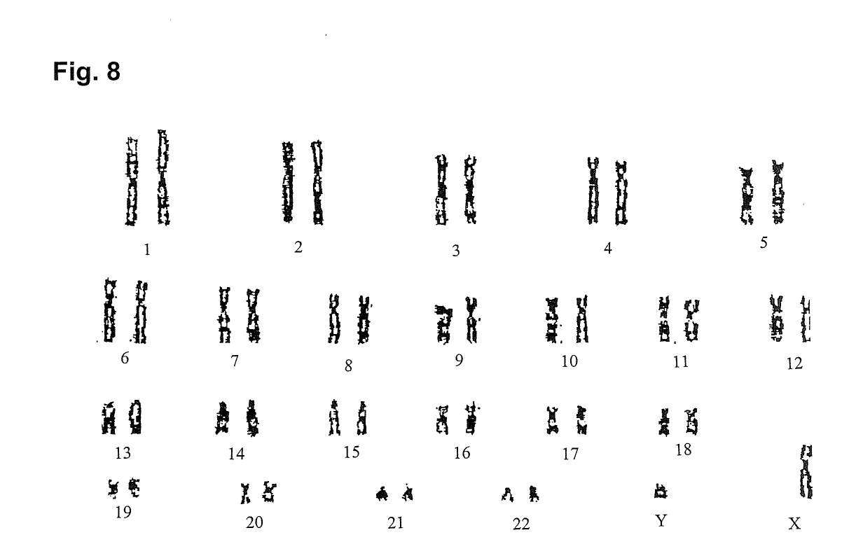

Devitrified human BS cells (line SA001) cultured on MEF were incubated in the presence of Calyculin A and then washed with cell culture medium. The cells were collected by centrifugation and fixed using ethanol and glacial acetic acid. The chromosomes were visualized using a trypsin-Giemsa staining. As illustrated in FIG. 8, the results show that there were no detectable chromosomal abnormalities in the cells following the vitrification and devitrification process.

### Telomerase Activity

For analyzing the telomerase activity a Telo TAGGG Telomerase PCR ELISA^{PLUS} kit (Roche, Basel, Switzerland) was employed according to the manufacturer's instructions. The assay uses the internal activity of telomerase, amplifying the product by PCR and detecting it with an enzyme linked immunosorbent assay (ELISA). Human BS cell line SA001 was analyzed after devitrification and culture on mouse embryonic feeder cells and displayed high telomerase activity. High telomerase activity in hBS cells correlates with their ability to divide indefinitely in culture.

### In Vitro Differentiation

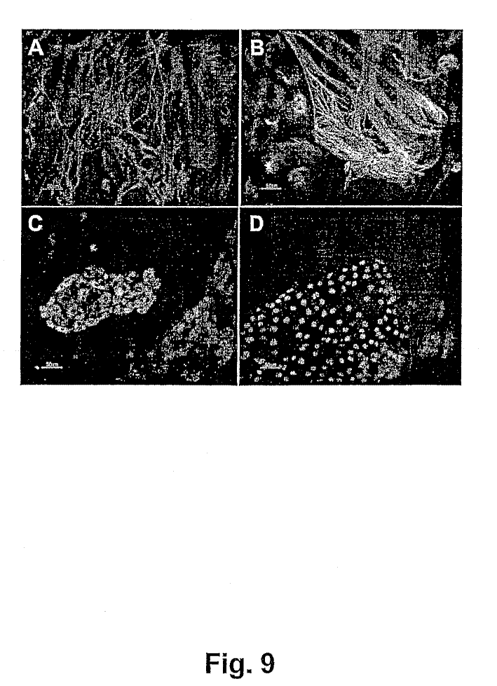

In order to investigate the pluripotency of devitrified human BS cells, undifferentiated colonies from cell line SA001 were transferred to suspension cultures using the Stem Cell Cutting Tool (Swemed Lab, Göteborg, Sweden) to allow the formation of embryoid bodies (EBs). Subsequently, EBs were plated in tissue culture plates and spontaneously differentiated cells were subjected to immunohistochemical evaluation using antibodies directed against β-III-tubulin (ectoderm), desmin (mesoderm), α-fetoprotein and HNF-3β (endoderm). Spontaneously contracting cells resembling cardiomyocytes were also observed (not shown). Taken together, these results show that human BS cells subjected to the vitrification and devitrification process retained their potential to differentiate into cells representing the three different germ layers in vitro (i.e., they remain pluripotent). The results are illustrated in FIG. 9.

### In Vivo Differentiation

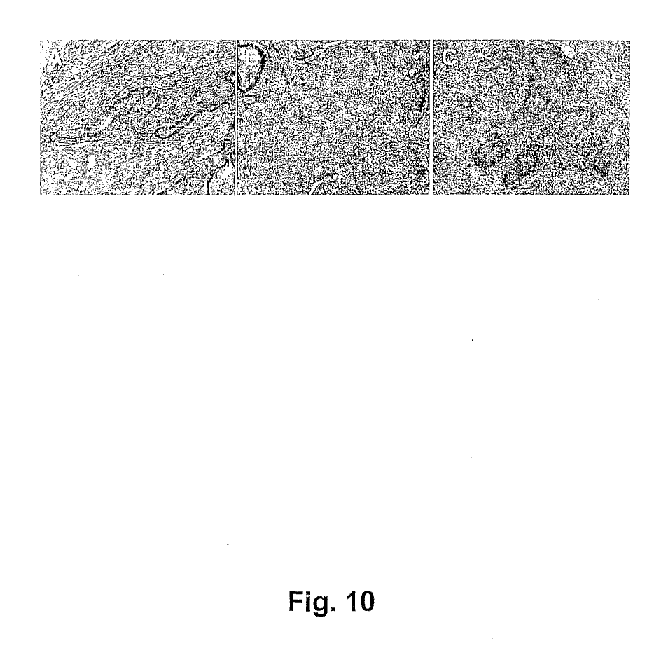

In order to investigate the pluripotency of devitrified human BS cells, undifferentiated cells (cell line SA001) were surgically placed under the kidney capsule of severe combined immuno-deficient (SCID) mice. The mice were sacrificed after 8 weeks and tumors were dissected and fixed in PFA. Histological evaluation of hematoxylin-eosin stained paraffin sections was performed in order to determine the presence of tissues derived from all three germ layers. As illustrated in FIG. 10, human BS cells subjected to the vitrification and devitrification process retained their potential to differentiate into cells representing the three different germ layers in vivo (i.e., they remain pluripotent).

A General Method for Establishment of Cells that can be Used in the Vitrification Procedure

Method for establishing hBS cells suitable for use in a method of the present invention

In PCT application published as WO 03/055992 (to the same Application) on 10 Jul. 2003, i.e. after the priority date of the present invention, a suitable method for establishing hBS cells is described. In one aspect of the present invention, the cells employed are obtained by the method claimed in WO 03/055992, which is hereby incorporated by reference.

The method for establishing pluripotent human blastocyst-derived stem cells or cell line from a fertilized oocyte comprises the steps of
i) using a fertilized oocyte optionally, having a grade 1 or 2, to obtain a blastocyst, optionally having a grade A or B,
ii) co-culturing the blastocyst with feeder cells for establishing one or more colonies of inner cell mass cells,
iii) isolating the inner cell mass cells by mechanical dissection,
iv) co-culturing of the inner cell mass cells with feeder cells to obtain a blastocyst-derived stem cell line.
v) optionally, propagation of the blastocyst-derived stem cell line.

As a starting material for this procedure, fertilized oocytes are used. The quality of the fertilized oocytes is of importance for the quality of the resulting blastocysts. The human blastocysts in step i) of the method may be derived from frozen or fresh human in vitro fertilized oocytes. In the following is described a procedure for selecting suitable oocytes for use in a method according WO 03/055992. It was found that an important success criterion for the present method is a proper selection of oocytes. Thus, if only grade 3 oocytes are applied, the probability of obtaining a hBS cell line fulfilling the general requirements (described below) is low.

Donated fresh fertilized oocytes: On day 0 the oocyte is aspirated in Asp-100 (Vitrolife), and fertilized on day 1 in IVF-50 (Vitrolife). The fertilized oocyte is evaluated based on morphology and cell division on day 3. The following scale is used for fertilized oocyte evaluation:

Grade 1 fertilized oocyte: Even blastomers, no fragments
Grade 2 fertilized oocyte: <20% fragments
Grade 3 fertilized oocyte: >20% fragments

After evaluation on day 3, fertilized oocytes of grade 1 and 2 are either implanted or frozen for storage. Fertilized oocytes of grade 3 are transferred to ICM-2 (Vitrolife). The fertilized oocytes are further cultured for 3-5 days (i.e. day 5-7 after fertilization). The blastocysts are evaluated according to the following scale:

Grade A Blastocyst: Expanded with distinct inner cell mass (ICM) on day 6
Grade B Blastocyst: Not expanded but otherwise like grade A
Grade C Blastocyst: No visible ICM

Donated frozen fertilized oocytes: At day 2 (after fertilization) the fertilized oocytes are frozen at the 4-cell stadium using Freeze-Kit (Vitrolife). Frozen fertilized oocytes are stored in liquid nitrogen. Informed consent is obtained from the donors before the 5-year limit has passed. The fertilized oocytes are thawed using Thaw-Kit (Vitrolife), and the procedure described above is followed from day 2.

As described above, fresh fertilized oocytes are from grade 3 quality, and frozen fertilized oocytes are from grade 1 and 2. According to data obtained by the establishment methods, the percentage of fresh fertilized oocytes that develop into blastocysts is 19%, while 50% of the frozen fertilized oocytes develop into blastocysts. This means that the frozen fertilized oocytes are much better for obtaining blastocysts, probably due to the higher quality of the fertilized oocytes. 11% of the blastocysts derived from fresh fertilized oocytes develop into a stem cell line, while 15% of the blastocysts derived from frozen fertilized oocytes develop into a stem cell line. In summary, of the fertilized oocytes that were put into culture 2% of fresh fertilized oocytes developed into a stem cell line, and 7% of frozen fertilized oocytes that were put into culture developed into a stem cell line.

The culturing of the fertilized oocyte to the blastocyst-stage is performed after procedures well-known in the art. Procedures for preparing blastocysts may be found in Gardner et al, Embryo culture systems, In Trounson, A. O., and Gardner, D. K. (eds), Handbook of in vitro fertilization, second edition. CRC Press, Boca Raton, pp. 205-264; Gardner et al, Fertil Steril, 74, Suppl 3, O-086; Gardner et al, Hum Reprod, 13, 3434, 3440; Gardner et al, J Reprod Immunol, In press; and Hooper et al, Biol Reprod, 62, Suppl 1, 249.

After establishment of blastocysts in step i) optionally derived from fertilized oocytes having grade 1 or 2, the blastocysts having grade A or B are co-cultured with feeder cells for establishing one or more colonies of inner cell mass cells. After being plated onto feeder cells, their growth is monitored and when the colony is large enough for manual passaging (approximately 1-2 weeks after plating), the cells may be dissected from other cell types and expanded by growth on new feeder cells. The isolation of the inner cell mass cells is performed by mechanical dissection, which may be performed by using glass capillaries as a cutting tool. The detection of the inner cell mass cells is easily performed visually by microscopy and, according, it is not necessary to use any treatment of the oocytes with enzymes and/or antibodies to impair or remove the trophectoderm.

Thus, the procedure of WO 03/055992 alleviates the need for immunosurgery. By comparing the success-rate in using immunosurgery versus the present method, which leaves the trophectoderm intact, it has been observed that the much simpler, faster and non-traumatic procedure of avoiding immunosurgery is more efficient than immunosurgery. These procedures make the preparation of stem cell lines, and the differentiation of these cell lines commercially feasible. From a total of 122 blastocysts, 19 cell lines were established (15.5%). 42 blastocysts were processed by immunosurgery and 6 of these resulted in successfully established cell lines (14%). Eighty blastocysts were processed by the present method and 13 cell lines were established (16%).

Subsequent to dissection of the inner cell mass, the inner cell mass cells are co-cultured with feeder cells to obtain a blastocyst-derived stem (BS) cell line. After obtaining the hBS cell line, the cell line is optionally propagated to expand the amount of cells. Thus, the blastocyst-derived stem cell line may be propagated e.g. by passage of the stem cell line every 4-5 days. If the stem cell line is cultured longer than 4-5 days before passage, there is an increased probability that the cells undesirably will differentiate.

A specific procedure of passaging the cells in a feeder culture system is given in Establishment example 5 herein.

Human BS cell lines may be isolated either from spontaneously hatched blastocysts or from expanded blastocysts with an intact zona pellucida. In the method described above the blastocyst in step i) is a spontaneously hatched blastocyst. For hatched blastocysts the trophectoderm may be left intact. Either hatched blastocysts or blastocysts with a removed or partially removed zona pellucida may be put on inactivated feeder cells.

Zona pellucida of the blastocyst may be at least partially digested or chemically frilled prior to step ii) e.g. by treatment with one or more acidic agents such as, e.g., ZD™-10 (Vitrolife, Gothenburg, Sweden), one or more enzymes or mixture of enzymes such as pronase.

A brief pronase (Sigma) treatment of blastocysts with an intact zona pellucida results in the removal of the zona. Other types of proteases with the same or similar protease activity as pronase may also be used. The blastocysts can be plated onto said inactivated feeder cells following the pronase treatment.

In an embodiment of the invention step ii) and/or step iv) may be performed in an agent that improves the attachment of the blastocysts and/or if relevant the inner cell mass cells to the feeder cells. A suitable substance for this purpose is a hyaluronic acid.

A suitable medium for plating the blastocysts onto feeder cells can be hBS-medium that may be complemented with hyaluronic acid, which seems to promote the attachment of the blastocysts on the feeder cells and growth of the inner cell mass. Hyaluronan (HA) is an important glycosaminoglycan constituent of the extracellular matrix in joints. It appears to exert its biological effects through binding interactions with at least two cell surface receptors: CD44 and receptor for HA-mediated motility (RHAMM), and to proteins in the extracellular matrix. The positive effects of HA during the establishment of hBS cells may be exerted through its interactions with the surfactant polar heads of phospholipids in the cell membrane, to thereby stabilize the surfactant layer and thus lower the surface tension of the inner cell mass or blastocyst which may result in increased efficiency in binding to the feeder cells. Alternatively, HA may bind to its receptors on the inner cell mass or blastocyst and/or to the feeder cells and exert biological effects which positively influence the attachment and growth of the inner cell mass. According to this, other agents that may alter the surface tension of fluids, or in other ways influence the interaction between the blastocyst and feeder cells can also be used in instead of hyaluronic acid.

In the method describe above culturing of the feeder cells is of importance for the establishment of the hBS cell line. The propagation of blastocyst-derived stem cell line may comprise passage of the feeder cells at the most 3 times, such as e.g. at the most 2 times.

Suitable feeder cells for use in a method of the invention are fibroblasts of e.g. embryonic or adult origin. In a method according to the invention the feeder cells employed in steps ii) and iv) are the same or different and originate from animal source such as e.g. any mammal including human, mouse, rat, monkey, hamster, frog, rabbit etc. Feeder cells from human or mouse species are preferred.

Another important criterion for obtaining an hBS cell line fulfilling the general requirements are the conditions under which the blastocysts are cultured. The blastocyst-derived stem cell line may accordingly by propagated by culturing the stem cells with feeder cells of a density of less than about 60,000 cells per cm², such as e.g. less than about 55,000 cells per cm², or less than about 50,000 cells per cm².

In a specific embodiment, the propagation of blastocyst-derived stem cell line comprises culturing the stem cells with feeder cells of a density of about 45,000 cells per cm². These values apply in those cases where mouse feeder cells are used and it is contemplated that a suitable density can be found for other types of feeder cells as well. Based on the findings of the present inventors, a person skilled in the art will be able to find such suitable densities. The feeder cells may be mitotically inactivated in order to avoid unwanted growth of the feeder cells.

The blastocyst-derived stem cell line obtained by the establishment method described above maintains selfrenewal and pluripotency for a suitable period of time and, accordingly it is stable for a suitable period of time. In the present context the term “stable” is intended to denote proliferation capacity in an undifferentiated state for more than 21 months when grown on mitotically inactivated embryonic feeder cells.

The stem cell line obtained by the establishment method described above fulfils the general requirements. Thus, the cell line
i) exhibits proliferation capacity in an undifferentiated state for more than 21 months when grown on mitotically inactivated embryonic feeder cells, and
ii) exhibits normal euploid chromosomal karyotype, and
iii) maintains potential to develop into derivatives of all types of germ layers both in vitro and in vivo, and
iv) exhibits at least two of the following molecular markers OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2, and
v) does not exhibit molecular marker SSEA-1 or other differentiation markers, and
vi) retains its pluripotency and forms teratomas in vivo when injected into immuno-compromised mice, and
vii) is capable of differentiating.

The undifferentiated hBS cells obtained by the method described above are defined by the following criteria; they were isolated from human pre-implantation fertilized oocytes, i.e. blastocysts, and exhibit a proliferation capacity in an undifferentiated state when grown on mitotically inactivated feeder cells; they exhibit a normal chromosomal karyotype; they express typical markers for undifferentiated hBS cells, e.g. OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2, and do not show any expression of the carbohydrate epitope SSEA-1 or other differentiation markers. Furthermore, pluripotency tests in vitro and in vivo (teratomas) demonstrate differentiation into derivatives of all germ layers.

According to the above, the method provides an essentially pure preparation of pluripotent human BS cells, which i) exhibits proliferation capacity in an undifferentiated state for more than 21 months when grown on mitotically inactivated embryonic feeder cells; ii) exhibits normal euploid chromosomal karyotype; iii) maintains potential to develop into derivatives of all types of germ layers both in vitro and in vivo; iv) exhibits at least two of the following molecular markers OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2 v) does not exhibit molecular marker SSEA-1 or other differentiation markers, and vi) retains its pluripotency and forms teratomas in vivo when injected into immuno-compromised mice, and vii) is capable of differentiating.

Procedures for the detection of cell markers can be found in Gage, F. H., Science, 287:1433-1438 (2000). These procedures are well known for the skilled person and include methods such as RT-PCR or immunological assays where antibodies directed against the cell markers are used. In the following, methods for detection of cell markers, hybridisation methods, karyotyping, methods for measuring telomerase activity and teratoma formation are described. These methods can be used to investigate whether the hBS cells obtained according to the establishment method fulfil the above-mentioned criteria.

### Immunohistochemistry

The hBS stem cells maintained in culture are routinely monitored regarding their state of differentiation. Cell surface markers used for monitoring the undifferentiated hBS cells are SSEA-1, SSEA-3, SSEA-4, TRA-1-60, TRA-1-81. Human BS stem cells are fixed in 4% PFA and subsequently permeabilized using 0.5% Triton X-100. After washing and blocking with 10% dry milk the cells are incubated with the primary antibody. After extensive washes the cell are incubated with the secondary antibody and the nuclei are visualized by DAPI staining.

### Alkaline Phosphatase

The activity of alkaline phosphatase is determined using a commercial available kit following the instructions from the manufacturer (Sigma Diagnostics).

### Oct-4 RT-PCR

The mRNA levels for the transcription factor Oct-4 is measured using RT-PCR and gene specific primer sets (5′-CGTGAAGCTGGAGAAGGAGAAGCTG (SEQ ID NO: 1), 5′-CAAGGGCCGCAGCTTACACATGTTC (SEQ ID NO: 2)) and GAPDH as housekeeping gene (5′-ACCACAGTCCATGCCATCAC (SEQ ID NO: 3), 5′-TCCACCACCCTGTTGCTGTA (SEQ ID NO: 4)).

### Fluorescence In Situ Hybridization (FISH)

In one round of FISH one or more chromosomes are being selected with chromosome specific probes. This technique allows numerical genetic aberrations to be detected, if present. For this analysis CTS uses a commercially available kit containing probes for chromosome 13, 18, 21 and the sex chromosomes (X and Y) (Vysis. Inc, Downers Grove, Ill., USA). For each cell line at least 200 nuclei are being analyzed. The cells are resuspended in Carnoy's fixative and dropped on positively charged glass slides. Probe LSI 13/21 is mix with LSI hybridization buffer and added to the slide and covered with a cover slip. Probe CEP X/Y/18 is mixed with CEP hybridization buffer and added in the same way to another slide. Denaturing is performed at 70° C. for 5 min followed by hybridization at 37° C. in a moist chamber for 14-20 h. Following a three step washing procedure the nuclei are stained with DAPI II and the slides analyzed in an invert microscope equipped with appropriate filters and software (CytoVision, Applied Imaging).

### Karyotyping

Karyotyping allows all chromosomes to be studied in a direct way and is very informative, both numerical and larger structural aberrations can be detected. In order to detect mosaicism, at least 30 karyotypes are needed. However, this technique is both very time consuming and technically intricate. To improve the conditions for the assay the mitotic index can be raised by colcemid, a synthetic analog to colchicin and a microtubule-destabilizing agent causing the cell to arrest in metaphase, but still a large supply of cells are needed (6×10⁶ cells/analysis). The cells are incubated in the presence of 0.1 μg/ml colcemid for 1-2 h, and then washed with PBS and trypsinized. The cells are collected by centrifugation at 1500 rpm for 10 min. The cells are fixed using ethanol and glacial acetic acid and the chromosomes are visualized by using a modified Wrights staining.

### Comparative Genomic Hybridization

Comparative genomic hybridization (CGH) is complementary to karyotyping. CGH gives a higher resolution of the chromosomes and is technically less challenging.

Isolated DNA is nicktranslated in a mixture of DNA, A4, Texas red-dUTP/FITC 12-dUTP, and DNA polymerase I. An agarose gel electrophoresis is performed to control the size of resulting DNA fragments (600-2000 bp). Test and reference DNA is precipitated and resuspended in hybridization mixture containing formamide, dextrane sulfate and SSC. Hybridization is performed on denatured glass slides with metaphases for 3 days at 37° C. in a moist chamber. After extensive washing one drop of antifade mounting mixture (vectashield, 0.1 μg/ml DAPI II) is added and the slides covered with cover slips. Slides are subsequently evaluated under a microscope and using an image analysis system.

### Telomerase Activity

Since a high activity has been defined as a criterion for hBS cells 6 the telomerase activity is measured in the hBS cell lines. It is known that telomerase activity successively decrease when the cell reaches a more differentiated state. Quantifying the activity must therefore be related to earlier passages and control samples, and can be used as a tool for detecting differentiation. The method, Telomerase PCR ELISA kit (Roche) uses the internal activity of telomerase, amplifying the product by polymerase chain reaction (PCR) and detecting it with an enzyme linked immunosorbent assay (ELISA). The assay is performed according to the manufacturer's instructions. The results from this assay shows typically a high telomerase activity (>1) for hBS cells.

The cell lines retain their pluripotency and forms teratomas in vivo when injected into immuno-compromised mice. In addition, in vitro these cells can form hBS cell derived bodies. In both of these models, cells characteristic for all germ layers can be found.

### Teratoma Formation in Immunodeficient Mice

One method to analyze if a human BS cell line has remained pluripotent is to xenograft the cells to immunodeficient mice in order to obtain tumors, teratomas. Various types of tissues found in the tumor should represent all three germlayers. Reports have showed various tissues in tumors derived from xenografted immunodeficient mice, such as striated muscle, cartilage and bone (mesoderm) gut (endoderm), and neural rosettes (ectoderm). Also, large portions of the tumors consist of disorganized tissue.

Severe combined immunodeficient (SCID)-mice, a strain that lack B- and T-lymphocytes are used for analysis of teratoma formation. Human BS cells are surgically placed in either testis or under the kidney capsule. In testis or kidney, hBS cells are transplanted in the range of 10 000-100 000 cells. Ideally, 5-6 mice are used for each cell line at a time. Preliminary results show that female mice are more post-operative stable than male mice and that xenografting into kidney is as effective in generating tumors as in testis. Thus, a female SCID-mouse teratoma model is preferable. Tumors are usually palpable after approximate 1 month. The mice are sacrificed after 1-4 months and tumors are dissected and fixed for either paraffin-or freeze-sectioning. The tumor tissue is subsequently analyzed by immunohistochemical methods. Specific markers for all three germlayers are used. The markers currently used are: human E-Cadherin for distinction between mouse tissue and human tumour tissue, α-smooth muscle actin (mesoderm), α-Fetoprotein (endoderm), and β-III-Tubulin (ectoderm). Additionally, hematoxylin-eosin staining is performed for general morphology.

The establishment method is described below in the following “establishment examples”. The examples are included herein for illustrative purposes only and are not intended to limit the scope of the invention in any way. The general methods described herein are well known to a person skilled in the art and all reagents and buffers are readily available, either commercially or easily prepared according to well-established protocols in the hands of a person skilled in the art. All incubations were in 37° C., under a CO₂ atmosphere.

One suitable medium used is termed “BS-cell medium” or “BS-medium” and may be comprised of; KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine, 100 μM β-mercaptoethanol, 4 ng/ml human recombinant bFGF (basic fibroblast growth factor).

Another suitable medium is “BS cell body medium”, this may be comprised as follows; KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine and 100 μM β-mercaptoethanol.

In the present context the term “stable” is intended to denote proliferation capacity in an undifferentiated state for more than 21 months when grown on mitotically inactivated embryonic feeder cells.

### ESTABLISHMENT EXAMPLES

### Establishment Example 1

### Establishment of an Essentially Pure Preparation of Undifferentiated Stem Cells from Spontaneously Hatched Blastocysts

Human blastocysts were derived from frozen or fresh human in vitro fertilized embryos. Spontaneously hatched blastocysts were put directly on feeder cells (EF) in hBS cell medium (KNOCKOUT Dulbecco's Modified Eagle's Medium, supplemented with 20% KNOCKOUT Serum replacement, and the following constituents at the final concentrations: 50 units/ml penicillin, 50 μg/ml streptomycin, 0.1 mM non-essential amino acids, 2 mM L-glutamine, 100 μM β-mercaptoethanol, 4 ng/ml human recombinant bFGF (basic fibroblast growth factor), supplemented with 0.125 mg/ml hyaluronic acid. After plating the blastocysts on the EF cells, growth was monitored and when the colony was large enough for manual passaging approximately 1-2 weeks after plating) the inner cell mass cells were dissected from other cell types and expanded by growth on new EF cells.

### Establishment Example 2

### Establishment of an Essentially Pure Preparation of Undifferentiated Stem Cells from Blastocysts with an Intact Zona Pellucida

For blastocysts with an intact zona pellucida, a brief pronase (10 U/ml, Sigma) incubation in rS2 (ICM-2) medium (Vitrolife, Gothenburg, Sweden) was used to digest the zona, after which the blastocyst was put directly on the EF cell layer in hBS medium supplemented with hyaluronic acid (0.125 mg/ml).

### Establishment Example 3

### Histo-Chemical Staining for Alkaline Phosphatase

The cells were harvested for RT-PCR and histological (alkaline phosphatase) and immunocytochemical analysis (see below). RNA isolation and RT-PCR. Total cellular RNA was prepared using Rneasy Mini Kit (Qiagen) according to the manufacturer's recommendations. The cDNA synthesis was carried out using AMV First Strand cDNA Synthesis Kit for RT-PCR (Roche) and PCR using Platinum Taq DNA Polymerase (Invitrogen). Histochemical staining for alkaline phosphatase was carried out using commercially available kit (Sigma) following the manufacturer's recommendations.

### Establishment Example 4

### Preparation and Culturing of hBS Cell Line

Mouse embryonic fibroblasts feeder cells were cultivated on tissue culture dishes in EMFI-medium: DMEM (Dulbecco's Modified Eagle's Medium), supplemented with 10% FCS (Fetal Calf Serum), 0.1 μM β-mercaptoehanol, 50 units/ml penicillin, 50 μg/ml streptomycin and 2 mM L-glutamine (GibcoBRL). The feeder cells were mitotically inactivated with Mitomycin C (10 μg/ml, 3 hrs). Human BS cell-colonies were expanded by manual dissection onto inactivated mouse embryonic fibroblasts feeder cells.

Human BS cells were cultured on mitotically inactivated mouse embryonic fibroblasts feeder cells in tissue culture dishes with hBS-cell medium: KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine, 100 μM β-mercaptoethanol, 4 ng/ml human recombinant bFGF (basic fibroblast growth factor). Seven days after passage the colonies were large enough to generate BS cell bodies.

BS cell colonies were cut with glass capillaries into 0.4×0.4 mm pieces and plated on non-adherent bacterial culture dishes containing BS cell body medium: KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine and 100 μM β-mercaptoethanol. The BS cell bodies, including cystic hBS cell bodies, formed over a 7-9-day period.

### Establishment Example 5

### Passage of hBS Cells

Before passage the hBS cells are photographed using a Nikon Eclipse TE2000-U inverted microscope (10× objective) and a DXM 1200 digital camera. Colonies are passaged every 4-5 days. The colonies are big enough to be passaged when they can be cut in pieces (0.1−0.3×0.1-0.3 mm). The first time the cells are passaged, they have grown for 1-2 weeks and can be cut in approximately four pieces.

The colonies are focused, one by one, in a stereo-microscope and cut in a checkered pattern according to the size above. Only the inner homogeneous structure is passaged. Each square of the colony is removed with the knife, aspirated into a capillary and placed on new feeder cells (with the maximum age of 4 days). 10-16 squares are placed evenly in every new IVF-dish. The dishes are left five to ten minutes so the cells can adhere to the new feeder and then placed in an incubator. The hBS medium is changed three times a week. If the colonies are passaged, medium is changed twice that particular week. Normally a “half change” is made, which means that only half the medium is aspirated and replaced with the equal amount of fresh, tempered medium. If necessary the entire volume of medium can be changed.

### Establishment Example 6

### Vitrification of hBS Cells

Colonies with the appropriate undifferentiated morphology from the cell line are cut as for passage. 100-200 ml liquid nitrogen is sterile filtered into a sufficient amount of cryotubes. Two solutions A and B are prepared (A: 800 μl Cryo PBS with 1 M Trehalose, 100 μl etylen glycole and 100 μl DMSO, B: 600 μl Cryo PBS with 1 M Trehalose, 200 μl etylen glycole and 200 μl DMSO) and the colonies are placed in A for 1 minute and in B for 25 seconds. Closed straws are used to store the frozen colonies. After the colonies have been transferred to a straw, it is immediately placed in a cryotube with sterile filtered nitrogen.

### Establishment Example 7

### Seeding of Embryonic Mouse Feeder (EMFi) Cells

The cells are inactivated with EMFi medium containing Mitomycin C by incubation at 37° C. for 3 hours. IVF-dishes are coated with gelatin. The medium is aspirated and the cells washed with PBS. PBS is replaced with trypsin to detach the cells. After incubation, the trypsin activity is stopped with EMFi medium. The cells are then collected by centrifugation, diluted 1:5 in EMFi medium, and counted in a Bürker chamber. The cells are diluted to a final concentration of 170K cells/ml EMFi medium. The gelatin in the IVF-dishes is replaced with 1 ml cell suspension and placed in an incubator. EMFi medium is changed the day after the seeding.

A Method for Efficient Transfer of hBS Cells from a Feeder-Supported to a Feeder-Free Culture System, and Long-Term Propagation of hBS Cells Under Feeder-Free Conditions

The hBS cells employed in the present invention may be cultured in a feeder-free culture system, which method is advantageous compared to the known methods in that the cells transferred are stable for at least up to 10 passages. Studies by Richards et al. showed that the hBS cell lines could not be propagated in an undifferentiated state for more than six passages on cell-free matrixes, including Matrigel™. However, the hBS cells were stable for up to 35 passages on Matrigel™, still expressing the markers for undifferentiated hBS cells, even after a cycle of freeze/thawing and growth rates remained roughly comparable. Furthermore, a significantly higher number of surviving colonies were observed two days after plating, when mechanical dissociation was compared with enzymatic dissociation. A critical step seems to bee the initial step for transfer of the hBS cells to a feeder-free culture system. Accordingly, below is described a method for transfer of hBS cells to a feeder-free culture system, wherein the hBS cells are mechanically cut from the feeder. In the Feeder-free examples herein, only the centre part of each colony was used, whereas in previous work by Xu et al., the whole colonies were detached by enzymatic treatment with the risk of contaminating the cultures with feeder cells. Furthermore, the use of enzymes, at the very delicate step of transferring the feeder cultured hBS cells to a feeder-free surface, may cause inactivation of important surface molecules involved in cell adhesion and growth. The major components in Matrigel™ are extracellular matrix proteins, like collagen type IV and laminin.

Activation of the cell surface integrins upon binding to extracellullar matrix proteins is believed to be a crucial step for the regulation of cell adhesion, survival and proliferation. For example, Integrin alpha 1 has a unique role among the collagen receptors in regulating both in vivo and in vitro cell proliferation in collagenous matrices. Laminin-specific receptors, possibly formed by Integrin α6 and β1 which are highly expressed by hBS cells, may also play a major role in the adhesion of hBS cell to the matrix surface. Thus, one possibility is that some of the important surface receptors for attachment or survival might be negatively affected by the rough initial Collagenase IV treatment before the cells have adapted to the new surface.

In the examples herein different techniques for the transfer of hBS cells to a feeder-free environment were investigated, either by mechanical or enzymatical dissociation, in regards to cell adhesion, survival rate and proliferation. Furthermore, the method was developed in order to facilitate long-term propagation and large-scale production of homogenous populations of undifferentiated hBS cells. The use of conventional cryopreservation techniques for freezing/thawing of the hBS cells was also examined.

### Transfer of hBS Cells to Feeder Free Propagation

Subsequent to dissection of the inner cell mass, the inner cell mass cells are co-cultured with feeder cells to obtain a blastocyst-derived stem (BS) cell line. After obtaining the hBS cell line, the cell line is optionally propagated to expand the amount of cells.

Before propagation of the hBS cells in a feeder-free system, the hBS cells may be transferred to a feeder-free system.

As mentioned herein before and as it is demonstrated in the Feeder-free examples a critical factor for the success in the propagation of the hBS cells is the method by which the hBS cells is transferred from a feeder culture system to a feeder-free culture system. Accordingly, the hBS cells must be transferred to the feeder-free culture system by mechanical dissection, which may be performed by using glass capillaries as a cutting tool. As shown in the examples herein, mechanical dissociation resulted in a much more efficient attachment of cells to the Matrigel™, a more rapid proliferation compared to the enzyme treated cultures, and the cells were much more stable during passages. Accordingly, the method for transferring the HS cells according to the invention does not require any enzymatic treatment. As seen in the examples herein, the cells cultured and proliferated under feeder-free conditions have a mitotic index that was similar to that of cells grown under feeder conditions.

The propagation of the blastocyst-derived stem cell line comprises culturing the stem cells under feeder cell free growth conditions, as culturing the hBS cells without feeder cells has a number of advantages, such as, e.g. there is no need for the ongoing production of feeder cells, the production of hBS cells may be easier to scale up to commercial production and there is no risk of DNA transfer or other infection risks from the feeder cells.

Thus, the transfer and propagation step under feeder free conditions may comprise the following steps of
a) transferring the blastocyst derived stem cells from feeder to feeder free culture by mechanical treatment.
b) optionally, culturing the blastocyst derived stem cells under feeder cell free growth conditions in a suitable growth medium and/or on a suitable support substrate, and
c) optionally, passaging the blastocyst derived stem cell line every 3-10 days by enzymatic and/or mechanical treatment.

Normally, all steps i)-iii) are included.

Transfer of hBS Cells from a Feeder Culture System to a Feeder-Free Culture System

The transfer step has been found to be a critical step as mentioned above. Accordingly, the transfer should be done by means of mechanically dissociation or mechanical dissection of the cells in the feeder culture system. This mechanical treatment may be done by means of any suitable cutting tool such as a tool having a sharpened end and a size that is appropriate for the cutting. The tool may be made of any suitable material such as, e.g., plastic or glass and an example of a suitable tool is a cutting tool that is a sterile sharpened glass capillary, with a 25 degree angle and a 200 or 300 micrometer lumen, designed for cutting, manipulation, and transfer of hBS colonies, or parts of hBS colonies. It is produced by Swemed Lab International AB, Billdal, Sweden.

The hBS cells to be transferred is a colony of hBS cells and pieces is cut from the centre of the colony and suspended in a suitable medium as cell clusters. The cell clusters are dissociated mechanically one or more times e.g. until the cell clusters have a size that is at least 50% such as, e.g., at the most about 40%, at the most about 30%, at the most about 20%, at the most about 10% or at the most about 5% of that of the original colony. The size is e.g. determined as the diameter of the cluster or colony, respectively.

In the feeder-free examples herein is given suitable conditions for the transfer process. These conditions may of course be varied within appropriate limits, which is within the knowledge of a person skilled in the art.

### Feeder-Free Example 1

### Preparation of Conditioned VitroHES™-Medium (k-VitroHES™-Medium) for Feeder Free Cultures

To prepare mEF cells for conditioning of VitroHES™-medium, a confluent monolayer of mEF cells (passage two) was Mitomycin C treated and seeded in a concentration of 59 000 cells/cm² in a gelatin (0.1%; Sigma) coated culture flask in Dulbecco's Modified Eagle Medium (D-MEM) supplemented with 1% Penicillin/Streptomycin (PEST; 10000 U/ml), 10% Fetal Bovine Serum (FBS) and 2 mM GLUTAMAX™-I Supplement (200 mM); all from GibcoBRL/Invitrogen, Carlsbad, Calif., USA. After a 24 hour incubation period and one wash with PBS (GibcoBRL/Invitrogen), the medium was discarded and replaced with VitroHES™-medium (0.28 ml/cm²) for a 24 hour conditioning period. The conditioned VitroHES™-medium (k-VitroHES™-medium) was collected every day up to three times from the same mEF culture (in passage two) and sterile filtered by using a 0.2 μm low protein binding filter (Sarstedt, Landskrona, Sweden). The k-VitroHES™-medium was used either fresh or after freezing at −20° C. and supplemented with 4 ng/ml of bFGF (GibcoRL/Invitrogen) prior to use. The k-VitroHES™-medium may be used for up to one week if stored at +4° C. When stored at −20° C. for up to two months, no sign of reduced bioreactivity could be detected upon usage.

### Feeder-Free Example 2

### Transferring of hBS Cell Lines to Feeder Free Growth Conditions

Initial hBS cell lines were maintained on Mitomycin C treated mouse feeders in 10-50 passages and cultured in VitroHES™-medium supplemented with 4 ng/ml of human basic fibroblast growth factor (bFGF).

Two different techniques were evaluated for transferring of the hBS cells from feeder culture to Matrigel™ coated plates, one with mechanical dissociation and one with collagenase treatment. The hBS cells were cut in square pieces, which represented the middle of the colony, by using a stem cell cutting tool (Swemed Lab AB, Billdal, Sweden), and carefully detached and transferred the cells to HBSS solution. The stem cell tool is a sterile sharpened glass capillary, with a 25 degree angle and a 200 or 300 micrometer lumen, designed for cutting, manipulation, and transfer of hBS colonies, or parts of hBS colonies. It is produced by Swemed Lab International AB, Billdal, Sweden.

Enzymatic Treatment with Collagenase (for Comparison)

After washing in HBSS the cell clusters were transferred to a Collagenase IV solution (200 U/ml; Sigma) for enzymatic dissociation. The cells were incubated for 30 minutes at 37° C. and 5% CO₂. During the incubation period, repeated mechanical dissociations with a pipette were performed and the dissociation process monitored in an inverted microscope. After the incubation period the cell suspension was pelleted (400 G for 5 minutes) and washed once in KnockOut™ D-MEM (GibcoBRL/Invitrogen) before being resuspended in k-VitroHES medium.

Mechanical Dissociation According to the invention

After washing in HBSS the cell clusters were carefully dissociated mechanically by using a 1-ml automatic pipette. The dissociation process was completed when the size of the cell clusters represented approximately 1/10- 1/20 of the original colonies (average of 20 000 cells/original colony) corresponding to the size of cell aggregates generated by Collagenase IV treatment, as described above After washing in HBSS the colonies were transferred to collagenase IV solution (200 U/ml) to start the enzyme dissociation.

For the two different techniques, the cells were seeded into four wells each and incubated at 37° C. in 5% CO₂. Each experiment was repeated four times, with the same amount of cells seeded each time. After two and six days the colony size and number was calculated.

### Results of Feeder-Free Example 1 and 2

To optimize the transferring of the hBS cultures from feeder to feeder-free conditions, two different techniques were evaluated; one with mechanical dissociation and one with enzymatic dissociation. Mechanical dissociation resulted in a more efficient attachment of cells to the Matrigel™ and a more rapid proliferation compared to the enzyme treated cultures. A significantly higher number of surviving colonies were observed two days after plating, when mechanical dissociation was compared with enzymatic dissociation (FIG. 5). The total area of all colonies generated on Matrigel™ after dissociation with the two different techniques, respectively, was compared (P<0.001). Furthermore, six days after plating the total colony area in the mechanically dissociated cultures were significantly increased compared with the enzymatically dissociated cultures (P=0.036).

### Feeder-Free Example 3

Culture and Passage of hBS cells cultured on Matrigel™

Four different cell lines SA 002, AS 038, SA 121 and SA 167 were used in all experiments. The cell lines were propagated on Matrigel™ for up to 35 passages and the morphological appearance and other hBS characteristics remained unaltered even after a cycle of freeze/thawing. All cultures consisted of well defined colonies of hBS cells without morphological signs of differentiation. After about 3-6 days the cells were passaged by taken away the medium and 1 ml of Collagenase IV (200 U/ml) solution was added to each well and incubated for 15-20 minutes. To facilitate cell detachment from the surface mechanical dissociation was performed followed by another 15 minutes of incubation. The cells were then washed, resuspended in k-VitroHES™medium and seeded at a split ratio of 1:2 to 1:6 onto Matrigel™. The hBS cultures were passaged every 5 to 6 days and the medium was changed every second to third day.

### Result of Feeder-Free Example 3

Observations were made that during passage of the hBS cells established on Matrigel™, enzyme treatment with Collagenase IV was needed to detach the colonies from the surface. Enzymatic treatment during passage was also found to give an increased proliferation rate after seeding, compared to mechanical dissociation.

### Feeder-Free Example 4

Cryopreservation and thawing of hBS cells cultured on Matrigel™

Four different cell lines SA 002, AS 038, SA 121 and SA 167 were treated with collagenase IV for 20-30 minutes to separate the cells from each other before freezing. After centrifugation the cells were transferred to freezing medium, which contains k-VitroHES™-medium containing 10% DMSO, 30% serum replacement and 4 ng/ml of bFgF, in a concentration of 1 million cells per ml freezing medium. The final cell suspension was a mixture of both single cells and cell clusters. The cryotubes (0.5-1.0 ml of cell suspension) were rapidly transferred to Nalgene freezing container for storages in −80° C. over night or at least for 2 hours before long-term storage in Liquid Nitrogen.

### Thawing of the hBS Cells

k-VitroHES™-medium has to be prepared and preheated before thawing the cells by placing the cryotubes in 37° water bath until all of the cell suspension was thawed. The cell suspension was transferred to the preheated medium for 5 minutes before centrifugation (400 G in 5 minutes). Matrigel™ thin layer coated (BD) wells were rehydrated by adding 1 ml of k-VitroHES™-medium to the wells and incubate 30 minutes in 37° C. The cell pellet was resuspended in k-VitroHES™-medium and transferred to either 24- or 6-well Matrigel™ plates.

### Feeder-Free Example 5

### Characterization of Feeder Free Cultured hBS Cells

All characterization experiments were performed after establishment on Matrigel™ and after a cycle of freeze/thaw.

Immunocytochemistry: The cultures were passaged as described above, seeded into 6- or 24-well Matrigel™ plates and cultured for six days before performing the immunostaining. The cultures were washed in PBS, fixed with 4% formaldehyde (HistoLab, Gothenburg, Sweden) for 15 minutes at room temperature and then washed again three times in PBS. The monoclonal primary antibodies used were directed against SSEA-1, -3 and -4 (1:200; Developmental Studies Hybridoma Bank, University of Iowa, Iowa City, Iowa), Tra-1-60, Tra-1-81 (1:200; Santa Cruz Biotechnology, Santa Cruz, Calif.), and polyclonal rabbit anti-Phospho-Histone H3 (1:150; KeLab, Upstate). The primary antibodies were incubated over night at 4° C. before being visualized using appropriate Cy3- or FITC-conjugated secondary:antibodies (1:300; Jackson ImmunoResearch Laboratories, West Grove, Pa.). Cultures were also incubated with 4′-6′Diamidino-2-phenylindole (DAPI; Sigma-Aldrich Sweden AB, Stockholm, Sweden), at a final concentration of 0.5 ug/mL for 5 minutes at room temperature, to visualize all the cell nuclei. The stained cultures were rinsed and mounted using DAKO fluorescent mounting medium (Dakopatts AB, Älvsjö, Sweden) and visualized in an inverted fluorescent microscope (Nikon Eclipse TE2000-U). Alkaline phosphatase (AP) staining of the Matrigel™ cultured hBS cells was carried out according to the manufacturer's instructions using a commercially available kit (Sigma-Aldrich).

Telomerase activity: Matrigel™ cultured hBS cells were harvested, lysed and telomerase activity analyzed by a PCR-based ELISA (Roche Diagnostics GmbH, Mannheim, Germany) according to manufacturers instructions.

Karyotyping and FISH: The Matrigel™ propagated hBS cells designated for karyotyping were incubated for 1 to 3 hours in colcemid (0.1 μg/ml, Invitrogen, Carlsbad, Calif., USA), dissociated, fixated, mounted on glass slides and the chromosomes visualized by using a modified Wrights staining (#WS-32, Sigma). Preparation of metaphase plates was performed as previously described. For the fluorescence in situ hybridization (FISH) analysis, a commercially available kit (MultiVysion™ PB Multicolour Probe Panel; Vysis, Inc., Downers Grove, Ill.) containing probes for chromosome 13, 18, 21 and the sex chromosomes (X and Y) was used according to the manufacturer's instructions. Slides were analyzed using an invert microscope equipped with appropriate filters and software (CytoVision, Applied Imaging, Santa Clara, Calif.).

Teratomas: For the teratoma formation experiment, immunodeficient SCID mice (C.B-17/IcrCrl-scidBR, Charles River Laboratories, Germany) were used. Matrigel™ propagated hBS colonies were enzymatically detached from the surface by using Collagenase IV (200 U/ml), mechanically dissociated into small cell aggregates and approximately 50 000 to 100 000 cells/organ were injected under the kidney capsule. Control animals were treated with Cryo-PBS injections or with primary brain cells from a littermate. The animals were sacrificed eight weeks after injection and the tumors were immediately fixed in a 4% solution of paraformaldehyde and paraffin embedded. For histological analysis the teratoma were sectioned to 8 μm and stained with Alcian Blue/Van Giesson.

RT-PCR analysis of Oct-4 expression: Total RNA was isolated from all four Matrigel™ cultured hBS cell lines by using RNeasy Mini Kit (Qiagen) according the manufacturer's instructions. The cDNA was synthesized from 1 μg of total RNA using AMV First Strand cDNA Synthesis Kit (Roche) and the PCR reaction preformed by using Platinum Taq DNA Polymerase (Invitrogen). The PCR reaction included four initial step-down cycles, with two repeated cycles for every annealing temperature, with denaturation for 15 seconds at 94° C., annealing temperature for 15 seconds at 660 to 60° C. and extension for 30 seconds at 72° C. The following cycles included 35 repeats with annealing temperature at 58° C. The forward and reverse primer sequences for Oct-4 were previously described. β-actin primers were used as internal controls (sense, 5′-TGGCACCACACCTTCTACAATGAGC-3′ (SEQ ID NO: 5); antisense, 5′-GCACAGCTTCTCCTTAATGTC-ACGC-3′ (SEQ ID NO: 6); 400 bp product). The PCR products were size fractioned by gel electrophoresis using a 1.5% agarose gel. Human liver was used as a positive control and water as negative control for the PCR reaction.

### Results of Feeder-Free Example 4 and 5

Cell lines SA 002, AS 038, SA 121 and SA 167 were frozen and thawed by using cryopreservation techniques to see if any changes in the characterization could be found. After thawing all four cell lines survived and started to grow on Matrigel™ coated plates in similar pattern

Pluripotency and maintenance of the four different hBS cell lines in feeder-free conditions was demonstrated and compared to previous results for feeder cultures of the respective cell lines. These characterizations were performed by examining the morphology, expression of undifferentiated markers, telomerase activity, karyotype, and differentiation in vivo.

Immunocytochemistry: SSEA-1 expression was negative in all feeder-free cultured hBS cell lines as opposed to staining with antibodies against SSEA-3, SSEA-4, TRA-1-60 and TRA 1-80 which show a clear positive immunoreaction as expected for pluripotent hBS cells. Further, the cells displayed high levels of AP reactivity in all four Matrigel™ propagated cell lines.

Telomerase activity: Analysis was preformed on three of the Matrigel™ cultured hBS cell lines (AS 038, SA 121 and SA 167). The hBS cells cultured on Matrigel™ were found to have high levels of telomerase activity.

Karyotyping and FISH: Karyotype analysis was preformed on two of the Matrigel™ cultured cell lines, AS 038 and SA 121. Three of three cells from cell line AS 038 and ten of twelve cells from cell line SA 121 were found to possess normal human 46, XY karyotype (FIG. 10). The remaining two cells from the SA 121 cell line expressed an abnormal karyotype of 45, XY and 42, XY. Although, karyotypic changes seem to be normal occurring events after prolonged culturing for both feeder and feeder-free hBS cell cultures. In this study karyotypic analysis of feeder cultured hBS cells were comparable with results after Matrigel™ propagation, suggesting that the hBS cell karyotype remains normal and stable under these feeder-free conditions. FISH analysis was performed on two of the Matrigel™ propagated cell lines (SA 121 (XY) and SA 167 (XX)). Analysis was performed for chromosomes X, Y, 18, 13 and 21. For both cell lines tested at least 93% were normal. The results from the FISH analysis were comparable with results from feeder cultured hBS cell lines.

Teratoma formation: Teratoma formation was performed for two Matrigel™ cultured hBS cell lines, SA 167 and SA 002, and the results showed that teratomas formed consisting of differentiated cells and tissue representative from all three germ layers (endoderm, mesoderm and ectoderm, providing evidence that the Matrigel™ propagated hBS cultures have retained their pluripotency.

Oct-4 expression: Oct-4 expression was high in all four cell lines cultured on Matrigel™.

### Feeder-Free Example 6

Comparison of mitotic index of hBS cells cultured under feeder-free conditions on Matrigel™ coated plates compared to hBS cells cultured on embryonic mouse feeder cells Cell line SA 121 was cultured in parallel under feeder-free conditions on Matrigel™ coated plates and on embryonic mouse feeder cells for 3 days. The number of cells in mitosis was then quantified by nuclear immunoreactivity for phosphorylated Histone H3. The mitotic index in both cultures was calculated in order to compare the growth rate between feeder-free and feeder cultured hBS cells,

### Result of Example 6

The mitotic index was similar in cultures grown under feeder-free (Matrigel™) compared to feeder layer conditions. The doubling time for the feeder-free cultures were roughly the same (around 35 hours) as for feeder propagated hES cells.

1. A method for vitrification of hBS cells or cells derived from hBS cells, comprising:
i) transfer of said cells to a first solution (solution A),
ii) optionally incubation of said cells in the first solution,
iii) transfer said cells obtained in step i) or ii) to a second solution (solution B),
iv) optionally incubation of said cells in the second solution,
v) transfer of at least 20 μl of said cells obtained from step iii) or iv) into one or more closed straws,
vii) sealing the one or more closed straws, and
vii) vitrification of the one or more closed straws.

2. A method according to claim 1, wherein the volume of said cells transferred in step v) is from about 20 μl to about 250 μl.

3. A method according to claim 1, wherein said hBS cells are hBS cell lines.

4. A method according to claim 1, wherein at least one of the first and second solutions comprises one or more cryoprotectants.

5. A method according to claim 4, wherein the one or more cryoprotectants is selected from the group consisting of glycerol, trehalose, sucrose, ethylene glycol, DMSO, propanediol, and or mixtures thereof.

6. A method according to claim 1, wherein the first and the second solution contain one or more cryoprotectants that are the same or different.

7. A method according to claim 4, wherein the concentration of the one or more cryoprotectants in the first and the second solution is the same or different.

8. A method according to claim 4, wherein the total concentration (calculated as % v/v, % w/w or M) of the cryoprotectant in the second solution is larger than that in the first solution.

9. A method according to claim 4, wherein the cryoprotectant is trehalose.

10. A method according to claim 9, wherein the concentration of trehalose is from about 0.02 M to about 1M.

11. A method according to claim 4, wherein the cryoprotectant is sucrose.

12. A method according to claim 11, wherein the concentration of sucrose is from about 0.02 M to about 1M.

13. A method according to claim 1, wherein at least one of the first and the second solution comprises a viscosity-adjusting agent.

14. A method according to claim 13, wherein the viscosity-adjusting agent is selected from the group consisting of Ficoll, Percoll, hyaluronic acid, albumin, polyvinyl pyrrolidone, alginic acid, gelatin and glycerol.

15. A method according to claim 13, wherein said viscosity-adjusting agent is Ficoll.

16. A method according to claim 15, wherein the concentration of Ficoll is at the most about 150 mg/ml.

17. A method according to claim 13, wherein the first and the second solution contain one or more viscosity-adjusting agents that are the same or different.

18. A method according to claim 13, wherein the concentration of the one or more viscosity-adjusting agents in the first and the second solution is the same or different

19. A method according to claim 1, wherein at least one of the first and second solutions is an aqueous solution.

20. A method according to claim 1, wherein step ii) is included.

21. A method according to claim 20, wherein the incubation is performed at about 37° C. for a time period from between 5 sec to about 20 min.

22. A method according to claim 1, wherein step iv) is included.

23. A method according to claim 22, wherein the incubation is performed at about 37° C. for a time period from between about 5 sec to about 10 min.

24. A method according to claim 22, wherein the incubation is performed at about 37° C. for about 30 sec or less.

25. A method according to claim 1, wherein about 50% or more of said cells are viable after being devitrified and cultured in a suitable medium.

26. A cell, which has undergone vitrification by the method defined in claim 1.

27. A method according to claim 1 further comprising devitrification by a method comprising:
viii) subjecting one or more vitrified closed straw to an environment having a temperature of from about room temperature to about 40° C. for a time period that allows the content of the closed straw to thaw,
ix) opening of the one or more closed straw,
x) subjecting said cells contained in the one or more opened closed straw to a washing procedure using a third solution (solution C).
xi) optionally transferring the washed cells obtained from step x) to a fourth solution (solution D), and
xii) optionally incubating said cells in the fourth solution,
xiii) optionally transferring said cells from xii) from the fourth solution and seeding said cells on feeder cells, and
xiv) optionally further cultivating said cells.

28. A method according to claim 27 comprising steps xi), xiii) and xiv).

29. A method according to claim 28, further comprising step xii).

30. A method according to claim 27, wherein the third and/or fourth (if relevant) solution comprises one or more cryoprotectants.

31. A method according to claim 30, wherein the one or more cryoprotectants is selected from the group consisting of glycerol, trehalose, sucrose, ethylene glycol, DMSO, propanodiol, and or mixtures thereof.

32. A method according to claim 31, wherein the one or more cryoprotectants is glycerol, trehalose, sucrose, or mixtures thereof.

33. A method according to claim 32, wherein the concentration of the cryoprotectant is from about 0.02 M to about 1 M.

34. A method according to claim 27, wherein the concentration of the cryoptotectant in the third solution is larger than the concentration of the cryoprotectant in the fourth solution, if relevant.


SEQUENCE LISTING (6 sequences)

SEQ ID NO: 1 - DNA, 25 bp, Artificial Sequence
  Primer
cgtgaagctggagaaggagaagctg

SEQ ID NO: 2 - DNA, 25 bp, Artificial Sequence
  Primer
caagggccgcagcttacacatgttc

SEQ ID NO: 3 - DNA, 20 bp, Artificial Sequence
  Primer
accacagtccatgccatcac

SEQ ID NO: 4 - DNA, 20 bp, Artificial Sequence
  Primer
tccaccaccctgttgctgta

SEQ ID NO: 5 - DNA, 25 bp, Artificial Sequence
  Primer
tggcaccacaccttctacaatgagc

SEQ ID NO: 6 - DNA, 25 bp, Artificial Sequence
  Primer
gcacagcttctccttaatgtcacgc